(19) United States
(12) Patent Application Publication
(10) Pub. No.: US 2005/0251346 A1
(43) Pub. Date: Nov. 10, 2005

(54) Method and apparatus for reaction route graphs for reaction mechanism and kinetics modeling

(76) Inventors: Ilie Fishtik, Worcester, MA (US); Caitlin A. Callaghan, Dover, NH (US); Ravindra Datta, Worcester, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/093,176

(22) Filed: Mar. 29, 2005

Related U.S. Application Data
(60) Provisional application No. 60/557,295, filed on Mar. 29, 2004.

(51) Int. Cl. C12Q 1/68; G06F 19/00; G01N 33/48; G01N 33/50; G01N 31/00

(52) U.S. Cl. 702/19; 702/22

(57) ABSTRACT

A method and apparatus for reaction route (RR) network analysis is provided in analogy with electrical networks and is based on the combined use of RR theory, graph theory, and Kirchhoff's laws. The result is a powerful new approach of “RR graphs” that is useful in not only topological representation of complex reactions and mechanisms but, when combined with techniques of electrical network analysis, is able to provide revealing insights into the mechanism as well as the kinetics of the overall reactions involving multiple elementary reaction steps including the effect of topological constraints. Unlike existing graph theory approaches of reaction networks, the present invention approach is suitable for linear as well as non-linear kinetic mechanisms and for single and multiple overall reactions. The methodology has broad applicability including atmospheric networks, metabolic networks, and catalytic reaction mechanisms.

### RELATED APPLICATION

This application claims the benefit of U.S. Provisional Application No. 60/557,295, filed on Mar. 29, 2004. The entire teachings of the above application are incorporated herein by reference.

### BACKGROUND OF THE INVENTION

This invention relates to the depiction and analysis of reaction mechanisms and kinetics of complex reaction systems in catalysis, biology and chemistry.

Reaction schematics of one kind or another are universally employed to depict reaction pathways in chemistry and cellular biology, and are invaluable in the study of reaction mechanisms. Typically, species, often showing molecular structure, are drawn and interconnected via arrows to show the reactions. Such a scheme, while well-suited for monomolecular reactions, becomes complicated when more than one species is involved in a reaction and especially when there are parallel pathways.

The term “reaction graphs” in the literature alludes to the topology of reaction mechanisms and derives from the fact that reaction schematics are structurally similar to graphs, with nodes or vertices denoting species, and branches or edges representing their reactions. The graph theoretical viewpoint of mechanisms, besides its intuitive appeal, allows the results of graph theory to be used for describing reaction topology, and is especially useful for computer assisted enumeration. The main use of chemical graph theory has been in the study of molecular structure; however, it is being increasingly utilized in the elucidation and generation of reaction mechanisms.

The structural depiction of complex reaction networks is crucial, since the structure affects function. Reaction schematics are, of course, universally employed. While these are invaluable in comprehension of reaction pathways, they are not typically amenable to quantitative analysis. Graph theory appears to be a natural tool for translating these drawings into a mathematical description. Indeed, graph theory has been extensively employed to address topological problems in chemistry, starting with, who investigated systematic enumeration of the structure of isomers of saturated hydrocarbons. The main use of graph theory in chemistry still remains in “chemical graphs,” i.e., the study and cataloging of molecular structure (Bonchev, D.; Rouvray, D. H. Chemical Graph Theory. Introduction and Fundamentals; Gordon & Breach Science: New York, 1991 and Trinajstic, N. Chemical Graph Theory; CRC Press: Boca Raton, FLa., 1983). However, it is being increasingly utilized in the elucidation (Balaban, A. T. Reaction Graphs. In Graph Theorectical Approaches to Chemical Reactivity; Bonchev, D., Menkenyan, O., Eds.; Kluwer Academic Pub.: Dordrecht, The Netherlands, 1194; pp 137 and Temkin, O. N.; Zeigamik, A. V.; Bonchev, D. Chemical Reaction Networks: A Graph-Theorectical Approach; CRC Press; New York, 1996) and computer generation of reaction mechanisms (Broadbelt, L. J.; Snurr, R. Q. Applied Catal. A: General 2000, 200, 23; Broadbelt, L. J.; Stark, S. M.; Klein, M. T. Comp. Chem. Eng. 1996, 20, 113; Fan, L. T.; Bertok, B.; Friedler, F. Comp. Chem. 2002, 26, 265; Ratkiewicz, A.; Truong, T. N. J. Chem. Inf Comp. Sci. 2003, 43, 36) Balaban, in Balaban, A. T.; Farcasiu, D.; Banica, R. Rev. Roum. Chim. 1966, 11, 1205 proposed “reaction graphs” for studying isomerization reactions. Since then, there have been numerous other reaction graph studies, as summarized in the book by Temkin et al. More recently, these methods have been applied to metabolic networks (Beard, D. A.; Liang, S.-D.; Qian, H. Biophys. J. 2002, 83, 79; Heinrich, R.; Schuster, S. The Regulation of Cellular Systems; Chapman and Hall: New York, 1996; Schilling, C. H.; Palsson, B. O. Proc. Natl. Acad. Sci. USA 1998, 95, 4193).

Unfortunately, the universal practice of depicting reaction intermediates individually as vertices or nodes connected by branches or edges restricts their application to linear kinetic mechanisms. Temkin et al. proposed the use of bipartite graphs for nonlinear kinetic mechanisms, i.e., those with elementary reactions with more than one intermediate on either side of the reaction. In their approach, one set of vertices on one side of the graph represents intermediates, while another set on the opposite side represents terminal species (i.e. the reactants and products). A third set of markers in the middle denotes elementary reaction steps. Arrows connect various species and reactions and their directions show whether species are being consumed or produced, with double arrows denoting non-unit stoichiometric coefficients. This, unfortunately, results in jumbled graphs that are devoid of intuitive appeal even for simple reactions.

Oster et al. (Oster, G. F., et al., Quart, Rev. of Biophys. 1973, 6, 1) and Oster and Perelson (Oster, G. F.; Perelson, A. L. IEEE Trans. Circuits and Sys. 1974, CAS-21, 709) utilized the analogy between reaction networks and electrical networks in their development of graph theory based “network thermodynamics.” They represented the topological structure of a reaction network as a directed graph, with species representing tree branches and links representing the reactions interconnecting the species. The forward and the reverse steps were represented separately by two links. The non-unit stoichiometric coefficient was represented by an ideal transformer. However, their chemical reaction network representation is particularly cumbersome, resulting in complicated networks, even for simple reaction systems. Surprisingly, few other researchers have quantitatively utilized the analogy between reaction systems and electrical circuits (Bockris, J. O. M.; Srinivasan, S. Fuel Cells: Their Electrochemistry; McGraw-Hill: New York, 1969); however, the results are either similarly cumbersome (Shiner, J. S. Adv. Therm. 1992, 6, 248) or only simple monomolecular examples have been treated.

### SUMMARY OF THE INVENTION

Applicants have developed a new graph-theoretical approach that overcomes many of the limitations of the current methodologies and is a powerful tool in the graphical depiction, as well as in mechanistic and kinetic interpretation, of reaction networks based on reaction route theory and their analogy with electrical circuits. These “reaction route graphs” are distinct from “reaction graphs” in that the nodes do not represent a given species but, rather, they simply show how the elementary steps, or graph branches, are connected to depict the various reaction routes. Thus, there is a direct analogy to conventional graphs. The approach has broad applicability including elementary reactions of arbitrary stoichiometry and multiple overall reactions.

With the advent of quantum mechanical calculations of catalytic molecular events and their energetics along with the availability of powerful semi-theoretical methods, (Shustorovich, E.; Sellers, H. Surf. Sci. Reports, 1998, 31, 1) such reaction pathway analyses will become increasingly indispensable. It is only a matter of time before we have a very fundamental understanding of the molecular steps involved in important catalytic processes along with their reliable kinetics. However, this is only the first step toward unraveling the mechanism and kinetics of the overall catalytic reactions of interest. The predicted steps must be organized into a coherent mechanism that illustrates the kinetics of the overall reaction. The objective of this invention is to develop such a framework.

There are currently available two alternate ways in which one might use the kinetic information on elementary reaction steps: 1) the conventional Langmuir-Hinshelwood-Hougen-Watson (LHHW) approach, in which an explicit rate expression might be derived based on the universal, but arbitrary, assumptions such as the rate-determining step (RDS), quasi-steady-state (QSS) approximation, most abundant reactive intermediate (MARI), etc., and 2) the so-called microkinetic approach, wherein no assumptions are made, but only numerical analysis of the resulting large system of ordinary or partial differential equations of intermediate and terminal species material balances for a given reactor configuration is possible. Although it does involve numerical solution, much information about the controlling steps and the reaction network can be discerned from microkinetic models.

In this complimentary approach (Fishtik, I.; Callaghan, C.; Datta, R. Reaction network analysis of the kinetics of water-gas-shift reaction on Cu(111). In Computational Material Science 15; Leszczynski, J., Ed. Elsevier, 2004), Applicants begin with a microkinetic analysis based on a priori predicted elementary reaction kinetics, but then utilize a systematic graph-theoretic approach in conjunction with reaction route theory and an analogy to electrical networks to elucidate the major pathways followed by systematic reduction of the network to arrive at simpler mechanisms including, when possible, precise rate expressions involving predicted rate constants. The reaction route approach developed earlier by Applicants (Callaghan, C. A.; Fishtik, I.; Datta, R.; Carpenter, M.; Chmielewski, M.; Lugo, A; Surf Sci. 2003, 541, 21; Fishtik, I.; Datta, R. Chem. Eng. Sci. 2000, 55, 4029; Fishtik, I.; Datta, R. Ind. Eng. Chem. Res. 2001, 40, 2416; Fishtik, I.; Datta, R. Surf. Sci. 2002, 512, 229) is a generalization of the conventional reaction route theory (Happel, J.; Sellers, P. H. Adv. Catal. 1983, 32, 273; Horiuti, J.; Nakamura, T. Adv. Catal. 1967, 17, 1; Milner, P. C. J. Electroanal. Soc. 1964, 111, 228; Temkin, M. I. The kinetics of some industrial heterogeneous catalytic reactions. In Advances in Catalysis; Eley, D. D., Pines, H., Weisz, P. B., Eds.; Academic Press: New York, 1979; Vol. 28; pp 173) and forms the basis of our work here.

This approach has applications in heterogeneous and enzyme catalyzed reactions, and may also be utilized for the analysis of non-catalytic reactions as well as the functioning of the cellular metabolic machinery. Further objects and advantages will become apparent from a consideration of the ensuing description and drawings.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a flow diagram illustrating one embodiment of the present invention for the purpose of constructing a reaction route (RR) graph and utilizing it for the reduction and simplification of a reaction mechanism.

FIG. 2 is the reaction route network illustrated conceptually as a mountain trek between two camps.

FIG. 3 consisting of FIG. 3A-3B, illustrates the translation of the RR graph into network. (A) An elementary reaction is viewed as a resistor, Rₚ=Aₚ/rₚ, connecting two nodes in a RR graph. The arrows indicate the direction of the forward reaction. (B) The affinity of the overall reaction (OR) is viewed as a voltage source. By the convention adopted here, the arrows indicate that the rates of the OR via the RR network and via the voltage source have opposite signs.

FIG. 4 illustrates both the analogy between Kirchhoff's Current Law and conservation of mass in terms of elementary reaction rates and the analogy between Kirchhoff's Voltage Law and thermodynamic consistency as applied to the elementary reaction affinities, as well as the translation of Ohm's Law into reaction parameters.

FIG. 5 tabulates the elementary reaction steps and their rate expressions for the DFHR System.

FIG. 6 lists the stoichiometrically distinct RRs for DFHS System.

FIG. 7 is the RR graph for the DHFR system presented in the first example.

FIG. 8 is the conventional kinetic graph for the DHFR system.

FIG. 9 illustrates the Δ-Y transformation of the RR graph to allow for simple evaluation of the overall resistance for the DHFR system.

FIG. 10 shows the overall rate vs. the simplified rate as a function of time for the DHFR reaction.

FIG. 11 consisting of FIG. 11A-11D, shows the following simulation results: (A) R₁₀+R₁₁ vs. R₁+R₆ as a function of time for the DHFR reaction in a batch reactor; (B) R₁₂+R₁₃ vs. R₃+R₉ as a function of time for the DHFR reaction in a batch reactor; (C) R₁+R₂ vs. R₃+R₄ as a function of time for the DHFR reaction in a batch reactor; and (D) R₆+R₇ vs. R₈+R₉ as a function of time for the DHFR reaction in a batch reactor.

FIG. 12 tabulates a 17-step microkinetic model for WGS reaction on Cu where the activation energies are estimated according to the UBI-QEP method of Shustorovich (REF) and the pre-exponential factors are estimated from transition state theory and then adjusted as to fit the thermodynamics of the overall reaction.

FIG. 13 provides a list of the stoichiometrically distinct RRs for WGS reaction on Cu.

FIG. 14 illustrates the non-minimal RR graph for the 17-step WGSR on Cu.

FIG. 15 shows the two equivalent minimal RR graphs resulting from deletion of steps s₁₃ and s₁₆ as two subgraphs of the RR graph in FIG. 14.

FIG. 16 demonstrates the stepwise reduction of the RR network based on the comparison of resistances of parallel pathways.

FIG. 17 illustrates the application of the RR graph to the depiction of an energy diagram for the simplified RR graph of the WGS reaction.

FIG. 18 validates the theoretical prediction (Eq.[24]) of WGS Activity of Cu.

FIG. 19 is a schematic diagram of a computer network environment in which the present invention may be embodied and practiced.

FIG. 20 is a block diagram of a computer of the network of FIG. 19.

### DETAILED DESCRIPTION OF THE INVENTION

A description of preferred embodiments of the invention follows.

The structure of reaction mechanisms and kinetics are a major occupation of many a scientist in the areas of chemistry, catalysis, molecular biology, environmental science, and combustion, etc. Much effort is expended in proposing, accepting, and refuting speculative mechanisms based on spectroscopic or other, evidence of reacting intermediates. More recently, it has become possible to predict the structure and energetics of intermediates and transition states based on ab initio methods. Despite these advances, however, the various pathways, their relative importance, and identification of bottleneck steps are based mainly on guesswork. Rate expressions are often derived based on these assumptions. If experimental kinetics are in accord with predictions, the guessed mechanism is deemed acceptable. Applicants central purpose here is to develop a rigorous approach for deducing, not guessing, reaction pathways and overall kinetics based on first principles determination of reaction intermediates and elementary reaction kinetics.

Here, a new reaction route graph theory (RRGT) (Fishtik, I.; Callaghan, C. A.; Datta, R. J. Phys. Chem. B 2004, 108, 5671; Fishtik, I.; Callaghan, C. A.; Datta, R. J. Phys. Chem. B 2004, 108, 5683; Fishtik, I.; Callaghan, C. A.; Datta, R. J. Phys. Chem. B 2005, 109, 2710) that is a powerful tool in the graphical and mathematical depiction, as well as in mechanistic and kinetic interpretation, of reaction networks is presented. These “reaction route graphs” are distinct from “reaction graphs” in that the nodes do not represent a given species but, rather, they simply show how the elementary steps, or graph branches, are connected to depict the various reaction routes (RRs), which overcomes many of the limitations of earlier approaches, and allows a direct adaptation to equivalent electrical circuits.

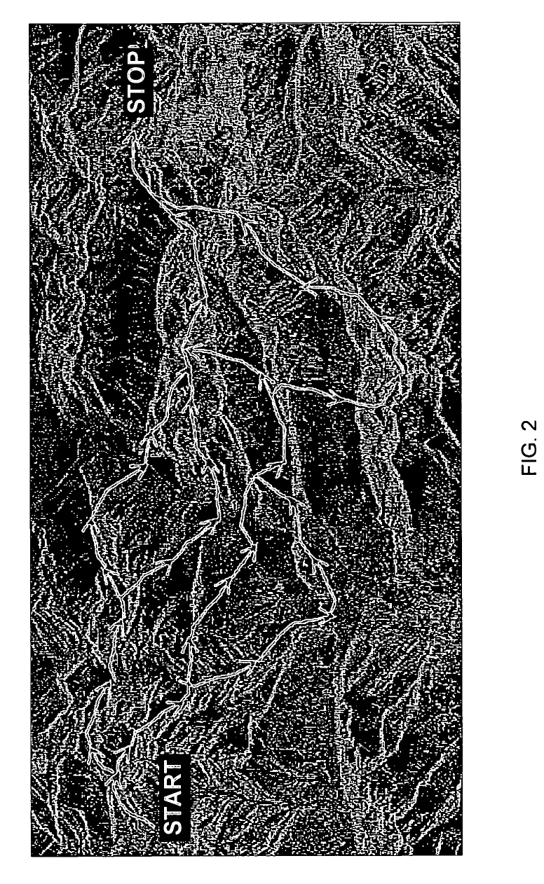

As a prelude to the present invention, it is useful to imagine a RR network as a trek across a mountain range with many peaks and valleys corresponding to individual elementary reaction steps involving a single reactive collision. FIG. 2 is illustrative.

In keeping with the transition-state theory, the valleys are viewed conceptually as the energy level of reactants or products, while an elementary reaction is viewed as a hike from one valley to an adjacent one over a mountain pass, representative of the energy barrier for the reaction. Many such excursions constitute the overall trek and correspond to the overall RR or ORR. Clearly, different hikers embarking on the trek would follow different routes. In fact, a lost hiker might go around in a cycle before making forward progress. Thus, an infinite variety of RRs exists, in principle, for a given trek in going from the reactants to the products, including those involving cycles, called empty reaction routes (ERRs). The direct RRs are defined as those that are the shortest, not involving any cycles. With this picture in mind, Applicants consider some definitions first followed by further details below.

Definition of Reaction Route Graph

A reaction route graph, G_{R}(s_{ρ}, nⱼ), is a directed graph composed of two sets, a set of B branches, or edges, representing reactions s_{ρ}, where s₀≡OR, while the s₁, s₂, . . . , sₚ are elementary reaction steps comprising the mechanism of OR, and a set of N nodes, or vertices, nⱼ, (j=1, 2, . . . , N), representing the interconnectivity of branches or reactions, so that the RRs, i.e., the pathways reactants take to form products, may be traced as trails, or walks, on G_{R}. G_{R}^{s}(s_{ρ}^{s}, nⱼ^{s}) is a subgraph of G_{R}(s_{ρ}, nⱼ) if s_{ρ}^{s} and nⱼ^{s} are, respectively, subsets of s_{ρ} and nⱼ. Each reaction branch s_{ρ}(nᵢ, nⱼ) is incident from node nᵢ and incident to node nⱼ. The number of branches incident on a node determines its degree d(nⱼ). Even though a branch may occur more than once within a G_{R}, they are distinguished by their set of end nodes, i.e., location. A branch is characterized by its affinity A_{ρ}(=−ΔG_{ρ}), akin to branch voltage, and rate, r_{ρ}, analogous to branch current. The A_{ρ} and r_{ρ} of a branch remain unchanged with location. Parallel branches have the same pair of end nodes and, thus, represent the same branch in parallel. A RR graph is minimal, G_{R,min,} if each branch occurs in it only once. Branches are drawn as lines with arrows showing assumed forward direction. The reaction may actually proceed in either direction, depending upon its A_{ρ}.

The incidence of branches at the nodes in G_{R} is given by the incidence matrix M=[m_{jρ}], with rows corresponding to the N nodes and columns to the B branches of the G_{R}. The elements of the incidence matrix are m_{jρ}=+1 if the branch s_{ρ}(nᵢ, nⱼ) is incident from node nⱼ, m_{jρ}=−1 if s_{ρ}(nᵢ, nⱼ) is incident to node nⱼ, and m_{jρ}=0 if the s_{ρ}(nᵢ, nⱼ) is not incident at nⱼ. Thus, a node may be represented by

$${n}_{j} \text{:} {\sum }_{ρ} {m}_{j ρ} {s}_{ρ} ( {n}_{i} , {n}_{j} ) .$$

Nodes may also be construed as representing a group of species and properties associated with them. In conventional graph theory, since every branch s_{ρ} of G_{R} is only incident to one node and incident from another node, each column of M has exactly one +1, one −1, the remainder elements being zero. However, since in RR graphs a branch or reaction may occur between more than one pair of nodes, there are corresponding entries of +1 and −1 at more than one pair of nodes in M. For parallel branches, the elements in M correspond to the number of parallel branches. However, the sum of each column in M=0, and, therefore, the rank M=N−1. Thus, any row, may be deleted to obtain the (N−1)×B reduced incidence matrix M_{f}. In M, the intermediate nodes (INs), n_{Ij}, are distinguished as those with only elementary steps incident on them, while terminal nodes (TNs), n_{Tj}, are those that also have OR.

The g^{th} trail, or walk, from starting node, nₛ, to ending node, nₑ, is an alternating sequence of nodes and branches, i.e.,

$${w}_{g} = {\sum }_{{n}_{s} \to {n}_{e}} {σ}_{g ρ} {s}_{ρ} ( {n}_{i} , {n}_{j} ) .$$

If the branch s_{ρ}(nᵢ, nⱼ) is oriented along the g^{th} trail, σ_{gρ}=+1, otherwise σ_{gρ}=−1. If a branch is not in it, σ_{gρ}=0. A given branch s_{ρ}(nᵢ, nⱼ) may not be traversed more than once in a trail, although a node may be crossed more than once. A closed trail begins and ends at the same node. A closed trail in which no node except the starting node appears more than once is a RR. If a RR includes the OR, it is an overall RR (or ORR); if only elementary reaction steps are traversed, it represents an empty RR (or ERR). The RR matrix σ=[σ_{gρ}] of a G_{R} is a matrix with rows corresponding to the RRs (ORRs and ERRS) and columns to the branches s_{ρ}(nᵢ, nⱼ). The number of independent RRs, L=B−N+1. The i^{th} submatrix of σ comprising a set of L linearly independent RRs is the fundamental RR matrix, σ_{f}⁽ⁱ⁾.

A (spanning) reaction tree T_{R} is a connected subgraph of G_{R} that has all of the nodes of the original G_{R} but is without some of its branches so that it has no RRs. Thus, T_{R} has N nodes and N−1 branches, called its twigs. Those branches of the G_{R} that are excluded in a given tree are its links. The number of twigs, i.e., the tree rank, is (N−1), and the number of links is L=B−N+1. Clearly, there are a large number of trees in a G_{R}, the i^{th} tree (i=1, 2, . . . , τ) being denoted by T_{R}⁽ⁱ⁾. If a link is added back to a T_{R}, the resulting graph contains one RR, which is assigned the orientation of the link. The addition of each subsequent link forms one or more additional RRs. The RRs that contain only one link are independent and called the fundamental RRs of the G_{R} corresponding to the i^{th} tree; these include at least one full ORR. There are, thus, L=B−N+1 findamental RRs corresponding to the i^{th} tree. This is represented by the fumdamental RR matrix of the i^{th} tree, σ_{f}⁽ⁱ⁾. If we further rearrange the columns in σ_{f}⁽ⁱ⁾ in the order of twigs followed by links, and rearrange the rows so that the first row corresponds to the fundamental RR in the first column, and so on, then σ_{f}⁽ⁱ⁾ takes the form: σ_{f}⁽ⁱ⁾=[σ_{f}⁽ⁱ⁾:I_{L}], where I_{L} is an identity submatrix of order L, and σ_{f}⁽ⁱ⁾ is the remaining submatrix corresponding to twigs of the i^{th} tree.

Network Conservation Laws

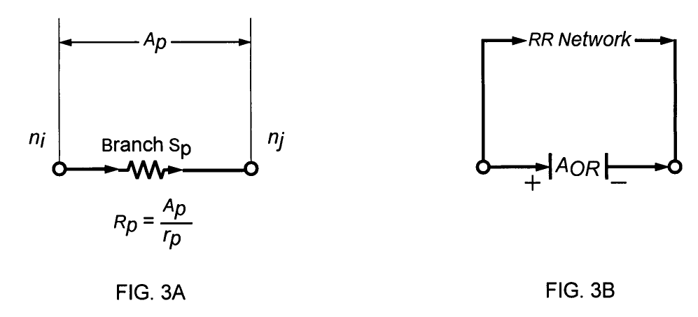

The RR graph can be directly converted into an equivalent electrical circuit by replacing each branch by its resistance R_{ρ}, defined later, and OR by a voltage source, A_{OR}, allowing utilization of Kirchhoff's laws. This is illustrated in FIG. 3A-3B.

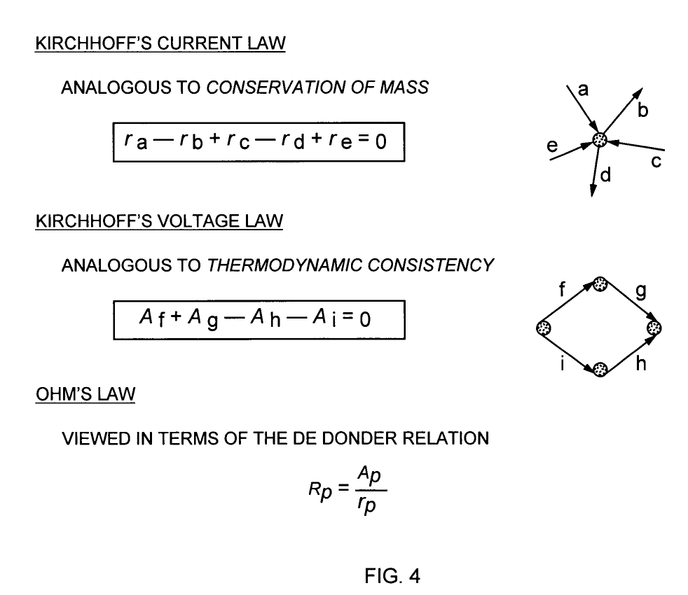

Kirchhoff's Current Law (Conservation of Mass): Assuming the node nⱼ to have a zero capacity, the net rates of reactions or branches (akin to branch current) incident at the node nⱼ sum to zero
M_{f}r=0 [1]
where r is a column matrix of branch rates, i.e., r≡(r₀ r₁ r₂ . . . rₚ)^{T}, the net rate being r_{ρ}=\vec{r}_{ρ}− [symbol] _{ρ}, along with the constitutive equation, r₀=−r_{OR}. Equation [1] is the result of the mass balance of a group of species around nⱼ, and is the equivalent of Kirchoff's current law (KCL). This is shown in FIG. 4.

Kirchhoff's Voltage Law, KVL (Thermodynamic Consistency): Being a state function, the sum of reaction affinities, akin to branch voltages, around the g^{th} RR (ORR or ERR) is zero, i.e.,
σ_{f}A=0 [2]
where A is a column matrix of branch affinities, A≡(A₀ A₁ A₂ . . . Aₚ)^{T}, with A₀=A_{OR}. Here, the affinity A_{ρ} for step s_{ρ}, i.e., the negative of the Gibbs free energy change (−ΔG_{ρ}), is related to the rates of forward and reverse steps by

$${A}_{ρ} ≡ \frac{{A}_{ρ}}{RT} = ln \frac{{\vec{r}}_{ρ}}{{\overleftarrow{r}}_{ρ}} \qquad [ 3 ]$$

where A_{ρ} is the dimensionless affinity. Combining Eqs. [2] and [3] provides

$$\begin{matrix}{\prod }_{{ERR}_{g}} {( {\vec{r}}_{ρ} / {\overleftarrow{r}}_{ρ} )}^{{σ}_{g ρ}} = 1 ; & and & {\prod }_{{ORR}_{g}} {( {\vec{r}}_{ρ} / {\overleftarrow{r}}_{ρ} )}^{{σ}_{g ρ}} = {\vec{r}}_{OR} / {\overleftarrow{r}}_{OR}\end{matrix} \qquad [ 4 ]$$

which is a thermodynamic constraint, as illustrated by FIG. 4, on kinetics of steps in a RR, with significant consequences.
RR Graph Topology from Reaction Stoichiometry

We consider the case of a single OR,

$${s}_{0} \text{:} {\sum }_{i} {v}_{i} {T}_{i} = 0 ,$$

where Tᵢ(i=1, 2, . . . , n) are the terminal species, i.e., reactants and products of the OR. The OR is, of course, the culmination of various elementary reaction steps s_{ρ}(ρ=1, 2, . . . , p) comprising the mechanism. These elementary reactions also involve intermediate species, Iₖ (k=1, 2, . . . l), that do not appear in the OR

$$\begin{matrix}{s}_{ρ} \text{:} & & {\sum }_{k = 1}^{l} {α}_{ρ k} {I}_{k} + {\sum }_{i = 1}^{n} {β}_{ρ i} {T}_{i} = 0 & & ( ρ = 1 , 2 , … , p )\end{matrix} \qquad [ 5 ]$$

The stoichiometric coefficient of intermediate Iₖ in reaction s_{ρ} is α_{ρk}, and that for terminal species Tᵢ is β_{ρi}. Only q of l intermediates are independent owing to some constraint (e.g., site balance). The affinity (De Donder and van Rysselberghe, 1936) for step s_{ρ}, in dimensionless form

$${A}_{ρ} = ln {K}_{ρ} - {\sum }_{k = 1}^{l} {α}_{ρ k} ln {a}_{k} - {\sum }_{i = 1}^{n} {β}_{ρ i} ln {a}_{i} \qquad [ 6 ]$$

where K_{ρ}=\vec{k}_{ρ}/ [symbol] _{ρ} is the equilibrium constant for the reaction s_{ρ}, and aᵢ is the species activity. Hence, the affinity may be calculated from the numerical results of a microkinetic analysis.

The overall stoichiometric matrix ν, with rows corresponding to reactions, including the OR as the first row, and the columns to the species, with intermediates followed by terminal species

$$v ≡ ⌊ \begin{matrix}0 & 0 & … & 0 & {v}_{1} & {v}_{2} & … & {v}_{n} \\ {α}_{11} & {α}_{12} & … & {α}_{1 l} & {β}_{11} & {β}_{12} & … & {β}_{1 n} \\ {α}_{21} & {α}_{22} & … & {α}_{2 l} & {β}_{21} & {β}_{22} & … & {β}_{2 n} \\ ⋮ & ⋮ & ⋮ & ⋮ & ⋮ & ⋮ & ⋮ & ⋮ \\ {α}_{p1} & {α}_{p2} & … & {α}_{pl} & {β}_{p1} & {β}_{p2} & … & {β}_{pn}\end{matrix} ⌋ \qquad [ 7 ]$$

In general, the rankν=q+1≦p. Thus, we define a reduced overall stoichiometric matrix γ for independent intermediate and terminal species, and a submatrix for independent intermediates α

$$\begin{matrix}γ ≡ ⌊ \begin{matrix}0 & 0 & … & 0 & {v}_{1} \\ {α}_{11} & {α}_{12} & … & {α}_{1 q} & {β}_{11} \\ {α}_{21} & {α}_{22} & … & {α}_{2 q} & {β}_{21} \\ ⋮ & ⋮ & ⋮ & ⋮ & ⋮ \\ {α}_{p1} & {α}_{p2} & … & {α}_{pq} & {β}_{p1}\end{matrix} ⌋ ; & α ≡ ⌊ \begin{matrix}0 & 0 & 0 & 0 \\ {α}_{11} & {α}_{12} & … & {α}_{1 q} \\ {α}_{21} & {α}_{22} & … & {α}_{2 q} \\ ⋮ & ⋮ & ⋮ & ⋮ \\ {α}_{p1} & {α}_{p2} & … & {α}_{pq}\end{matrix} ⌋\end{matrix} \qquad [ 8 ]$$

Since the intermediate species in α are linearly independent, rankα=q≦p. The quasi-steady state (QSS) condition for independent intermediate and terminal species is
Q≡γ^{T}r=0 [9]
where the column matrix for QSS condition of intermediates Qₖ and that for terminal species T₁, Q₀, is Q≡(Q₁ Q₂ . . . Q_{q} Q₀)^{T}. The QSS condition for only intermediates is given by Q=α^{T}r=0.

Overall Reaction Routes (ORRs): An ORR is defined as a linear combination of the elementary reaction steps, s₁, s₂, . . . , sₚ, that eliminates all the surface intermediates, thus resulting in the OR, i.e.,

$${ORR}_{g} \text{:} {\sum }_{ρ = 1}^{p} {σ}_{g ρ} {s}_{ρ} = OR ,$$

where σ_{gρ} is the stoichiometric number of reaction step s_{ρ} in the g^{th} RR. The use of Eq. [5] in this provides:

$${\sum }_{ρ = 1}^{p} {α}_{ρ k} {σ}_{g ρ} = 0 ;$$

(k=1, 2, . . . , l), or in matrix form
α^{T}σ_{g}=0 [10]
along with

$${\sum }_{ρ = 1}^{p} {β}_{ρ i} {σ}_{g ρ} = {v}_{i} ;$$

(i=1, 2, . . . , n), which provides v; once the values of σ_{gρ} have been determined from Eq. [10]. If all νᵢ=0, the resulting RR is an ERR. Here σ_{g} is the vector of stoichiometric numbers for the g^{th} RR. Since rankα=q, clearly only q unknown σ_{gρ}'s can be found uniquely by solving Eq. [10]. According to Milner, a direct ORR involves no more than rankα+1=q+1 linearly independent steps. Thus, the first q steps, along with the OR, may be viewed as twigs of a tree of RR graph, while the (q+1)^{st} step is a link. If a different (q+1)^{st} step (g^{th} link) is picked, a different RR_{g} (ORR_{g} or ERR_{g}) results. Further, since several linearly independent sets of q+1 steps from among p are possible, for the i^{th} such set in which the steps are renumbered such that OR, i.e., s₀, and s₁⁽ⁱ⁾,s₂⁽ⁱ⁾, . . . , s_{q}⁽ⁱ⁾ are twigs, while s_{q+1}⁽ⁱ⁾,s_{q+2}⁽ⁱ⁾, . . . , s_{q+g}⁽ⁱ⁾, . . . , sₚ⁽ⁱ⁾ are links, ORR_{g}⁽ⁱ⁾:

$${\sum }_{ρ = 1}^{q + 1} {σ}_{g ρ}^{( i )} {s}_{ρ}^{( i )} = OR .$$

The solution to Eq. [10] for σ_{gρ}⁽ⁱ⁾ thus provides

$${ORR}_{g}^{( i )} \text{:} OR = \frac{1}{{Δ}^{( i )}} \vert \begin{matrix}{α}_{11}^{( i )} & {α}_{12}^{( i )} & ⋯ & {α}_{1 q}^{( i )} & {s}_{1}^{( i )} \\ {α}_{21}^{( i )} & {α}_{22}^{( i )} & ⋯ & {α}_{2 q}^{( i )} & {s}_{2}^{( i )} \\ ⋮ & ⋮ & ⋯ & ⋮ & ⋮ \\ {α}_{q1}^{( i )} & {α}_{q2}^{( i )} & ⋯ & {α}_{qq}^{( i )} & {s}_{q}^{( i )} \\ {α}_{q + g , 1}^{( i )} & {α}_{q + g , 2}^{( i )} & ⋯ & {α}_{q + g , q}^{( i )} & {s}_{q + g}^{( i )}\end{matrix} \vert \qquad [ 11 ]$$

where Δ⁽ⁱ⁾ is the determinant of the matrix formed by the first q rows and columns of the matrix in Eq. [11]. By picking links one at a time from the remaining L=p−q branches, this povides the complete fimdamental RR matrix in the form σ_{f}⁽ⁱ⁾=[σₜ⁽ⁱ⁾ [symbol] I_{L}] for the corresponding RR graph.

Empty Reaction Routes (ERRs): A RR that eliminates all species, both intermediate and terminal, i.e., producing an OR in which all νᵢ=0, is an ERR. Although the majority of the ERRs are enumerated by the ORR algorithm above (i.e., for νᵢ=0), the following algorithm ensures a complete listing of ERRs. In analogy with direct ORRs, we define direct ERRs as those that involve no more than the rankγ+1=q+2 linearly independent steps. For the j^{th} independent set of q+2 elementary reactions in which the steps are renumbered such that s₁^{(j)},s₂^{(j)}, . . . , s_{q}^{(j)},s₊₁^{(j)} are the twigs, and s_{q+2}^{(j)},s_{q+3}^{(j)}, . . . , s_{q+g}^{(j)}, . . . , sₚ^{(j)} are links, the direct ERR becomes,

$${ERR}_{g}^{( j )} \text{:} {\sum }_{ρ = 1}^{q + 2} {σ}_{g ρ}^{( j )} {s}_{ ρ}^{( j )} = 0.$$

Substitution of Eq. [5] in this provides γ^{T}σ_{g}=0. A solution to this for σ_{gρ}^{(j)} results in

$${ERR}_{g}^{( j )} \text{:} \frac{1}{{D}^{( j )}} \vert \begin{matrix}{α}_{11}^{( j )} & {α}_{12}^{( j )} & ⋯ & {α}_{1 q}^{( j )} & {β}_{11}^{( j )} & {s}_{1}^{( j )} \\ {α}_{21}^{( j )} & {α}_{22}^{( j )} & ⋯ & {α}_{2 q}^{( j )} & {β}_{21}^{( j )} & {s}_{2}^{( j )} \\ ⋮ & ⋮ & ⋯ & ⋮ & ⋮ & ⋮ \\ {α}_{q1}^{( j )} & {α}_{q2}^{( j )} & ⋯ & {α}_{qq}^{( j )} & {β}_{q1}^{( j )} & {s}_{q}^{( j )} \\ {α}_{q + 1 , 1}^{( j )} & {α}_{q + 1 , 2}^{( j )} & ⋯ & {α}_{q + 1 , q}^{( j )} & {β}_{q + 1 , 1}^{( j )} & {s}_{q + 1}^{( j )} \\ {α}_{q + g , 1}^{( j )} & {α}_{q + g , 2}^{( j )} & ⋯ & {α}_{q + g , q}^{( j )} & {β}_{q + g , 1}^{( j )} & {s}_{q + g}^{( j )}\end{matrix} \vert = 0 \qquad [ 12 ]$$

where D^{(j)} is the determinant of the matrix formed by the first q+1 rows and columns of the matrix in Eq. [12]. By picking the links one at a time from the remaining p−q−1 branches, this provides the j^{th} ERR set. When all the trees are thus chosen, a complete listing of ERRs results.

Intermediate Nodes (INs): By definition, INs involve only elementary steps, i.e.,

$${n}_{Ij} \text{:} {\sum }_{ρ = 1}^{ρ} {m}_{j ρ} {s}_{ρ} .$$

Further, we define direct INs as those with the minimum degree, Thus, if a step is removed, it is no longer possible to satisfy the QSS condition Qⱼ for the the node by combining remaining rates. In fact, the QSS condition may be used to determine the connectivity of a node. Let Qⱼ be given by a linear combination of QSS for the individual species (Eq. [9]), i.e.,

$${Q}_{j} ≡ {\sum }_{k = 1}^{q} {Q}_{k} {λ}_{jk} = 0.$$

The use of Q=α^{T}r=0 in this for Qₖ, followed by a comparison with Eq. [1] results in
αλⱼ=mⱼ^{t} [13]
where mⱼ=(m_{jρ}) is j^{th} row of the incidence matrix, M, and provides the reaction steps incident on node nᵢⱼ. In the spirit of directness, we would like to find the multipliers λⱼₖ by solving Eq. [13] such that as many of m_{jρ}=0 as possible. Since the rankα=q, a nontrivial solution can be found only if no more than q−1 quantities m_{jρ}=0, i.e., no more than p−q+1=L+1 of the remaining steps with non-zero m_{jρ} are incident on nᵢⱼ. Thus, assuming that steps of the l^{th} independent set are labeled so that s₁^{(l)},s₂^{(l)}, . . . , s_{q-1}^{(l)} are not incident on the j^{th} node, while s_{q}^{(l)},s_{q+1}^{(l)}, . . . , s_{q+g}⁽ⁱ⁾, . . . , sₚ⁽ⁱ⁾ are incident on it, and re-labeling the latter as s₁^{(h)},s₂^{(h)}, . . . , sᵤ^{(h)}, . . . , s_{μ+1}^{(h)}, the solution to Eq. [13] provides

$${n}_{Ij} \text{:} {\sum }_{u = 1}^{μ + 1} \frac{1}{{δ}^{( l )}} \vert \begin{matrix}{α}_{11}^{( l )} & {α}_{21}^{( l )} & ⋯ & {α}_{q - 1 , 1}^{( l )} & {α}_{u1}^{( h )} \\ {α}_{12}^{( l )} & {α}_{22}^{( l )} & ⋯ & {α}_{q - 1 , 2}^{( l )} & {α}_{u2}^{( h )} \\ ⋮ & ⋮ & ⋯ & ⋮ & ⋮ \\ {α}_{1 , q - 1}^{( l )} & {α}_{2 , q - 1}^{( l )} & ⋯ & {α}_{q - 1 , q - 1}^{( l )} & {α}_{u , q - 1}^{( h )} \\ {α}_{1 q}^{( l )} & {α}_{2 q}^{( l )} & ⋯ & {α}_{q - 1 , q}^{( l )} & {α}_{uq}^{( h )}\end{matrix} \vert {s}_{u}^{( h )} \qquad [ 14 ]$$

where δ^{(l)} is obtained from the determinant in Eq. [14] by deleting the last row and the last column. By selecting the sets of q−1 steps excluded from the node one at a time from among the p reactions, the complete set of direct intermediate nodes can be obtained.

Terminal Nodes (TNs): The TNs are defined as nodes on which both elementary steps and OR are incident, i.e., n_{Tj}:

$${m}_{j0} ( OR ) + {\sum }_{ρ = 1}^{p} {m}_{j ρ } {s}_{ρ} .$$

The QSS conditions for terminal nodes provide γλⱼ=mⱼ^{T}, the solution of which for λⱼₖ provide the set of steps s₁^{(l)},s₂^{(l)}, . . . , s_{q}^{(l)} that are not incident on the j^{th} terminal node, with the remaining L steps s₁^{(h) ,s}₂^{(h)}, . . . , sᵤ^{(h)}, . . . , s_{L}^{(h)} being incident on it, in addition to the OR. Then the connectivity of j^{th} terminal node is given by

$${n}_{Tj} \text{:} {v}_{1} ( OR ) {\sum }_{u = 1}^{μ} \frac{1}{{Δ}^{( l )}} \vert \begin{matrix}{α}_{11}^{( l )} & {α}_{21}^{( l )} & ⋯ & {α}_{q1}^{( l )} & {α}_{u1}^{( h )} \\ {α}_{12}^{( l )} & {α}_{22}^{( l )} & ⋯ & {α}_{q2}^{( l )} & {α}_{u2}^{( h )} \\ ⋮ & ⋮ & ⋯ & ⋮ & ⋮ \\ {α}_{1 , q - 1}^{( l )} & {α}_{2 , q - 1}^{( l )} & ⋯ & {α}_{qq}^{( l )} & {α}_{uq}^{( h )} \\ {β}_{11}^{( l )} & {β}_{21}^{( l )} & ⋯ & {β}_{q1}^{( l )} & {β}_{u1}^{( h )}\end{matrix} \vert {s}_{u}^{( h )} \qquad [ 15 ]$$

Thus, by identifying all sets of q independent steps that are not incident on n_{Tj}, or alternately the L steps that may be incident on it, all the TNs are enumerated.
Construction of the RR Graph

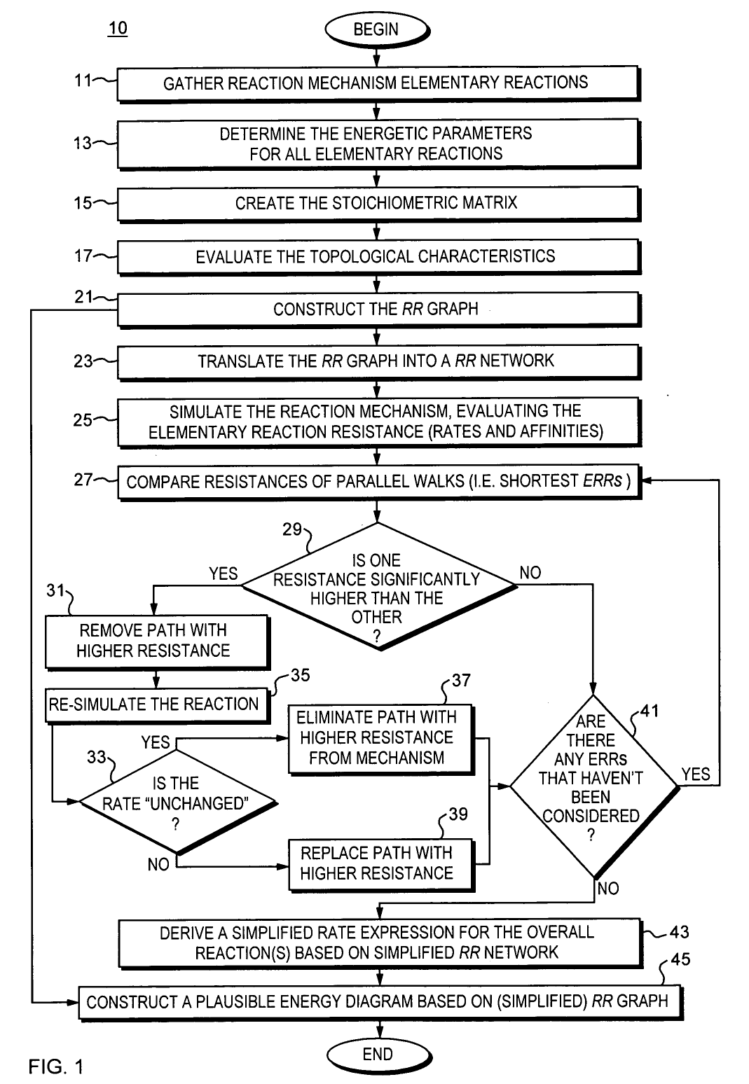

FIG. 1 is a flow diagram illustrating one embodiment of the present invention 10 for the purpose of constructing a RR graph and utilizing it for the reduction and simplification of a reaction mechanism.

In a first step 11, the illustrated embodiment 10 gathers reaction mechanisms of subject given elementary reactions, s₁, s₂, . . . sₚ. In step 13, the process determines the energetic parameters for all the subject elementary reactions. In step 15, the stoichiometric matrix (ν, with σ and M) is created, and the topological characteristics are evaluated at step 17. Construction of the RR graph (step 21) is then as follows.

If the elements σ_{gρ} of the matrix σ_{f}⁽ⁱ⁾ and m_{jρ} of the matrix M_{f} all comprise of only +1, −1, or 0, then the mechanism is amenable to the construction of minimal graphs, G_{R,min}. In these graphs, which are unique and direct, each elementary step s_{ρ} as well as OR occur only once. A direct RR graph is one in which all ORRs and ERRs that can be traversed as trails are direct. Thus, the circumference (length of the longest RR) of a direct G_{R}=q+2.

A non-minimal graph is one in which it is not possible to obtain matrices σ_{f}⁽ⁱ⁾ and M_{f} with only +1, −1, or 0. For example, a step for which σ_{gρ}=±2 must occur at more than one location, in accord with the definition of RRs as trails. In this case, the RR graphs are then symmetrical and involve every elementary step as well as OR exactly twice. This, of course, is appropriate as it preserves the rate and the affinity of a step s_{ρ} irrespective of location. This motivates the following construction process. We start with a σ_{f}⁽ⁱ⁾ matrix one of shortest ORRs with unit σ_{gρ}'s, the remaining being the shortest ERRS. The ORR is drawn with a sequence of steps that might be meaningful mechanistically, although the order is inconsequential from a kinetics viewpoint. Next, another version is drawn in which the sequence, but not the direction, of steps is reversed. The two ORRs are placed as subgraphs so that they have rotational, or point, symmetry of 180°. Thereafter, the ERRs are placed, one at a time, once each in each subgraph such that no step is repeated within a subgraph. The two subgraphs are then interconnected by fusing selected nodes from each subgraph only when the remaining ERRs cannot be contained entirely within a subgraph. Two nodes are fused when they are replaced by a single node containing all branches incident on the original nodes. Of course, the fused nodes must also be direct. When all of the remaining ERRs in σ_{f}⁽ⁱ⁾ have thus been incorporated, the OR has been included, and the resulting INs and TNs are among the enumerated nodes, the RR graph is complete. Finally, it is checked to ensure that the complete list of RRs can be traced on it as trails.

Electrical Circuit Analogy (Step 23)

Although the electrical analogy is commonly invoked to represent parallel and series steps in mechanisms, it is used mainly as a tool for comprehension. The exception is for impedance analysis of electrochemical reactions, where equivalent circuits are utilized quantitatively. The quantitative application of electrical analogy for general kinetic mechanisms here is, thus, new. In Step 23 of FIG. 1, each branch in the RR graph is replaced by its resistance R_{ρ} (for the steady-state case, or by a combination of resistance, capacitance, and inductance, for the unsteady-state case), while the branch representing the OR is replaced by a voltage source, A_{OR}. The resistance R_{ρ} is defined as the mean value of the 1/r_{ρ} between its limiting values, namely, \vec{r}_{ρ} and [symbol] _{ρ}

$${R}_{ρ} ≡ \frac{1}{{\vec{r}}_{ρ} - {\overleftarrow{r}}_{ρ}} {\int }_{{\overleftarrow{r}}_{ρ}}^{{\vec{r}}_{ρ}} \frac{1}{{r}_{ρ}} ⅆ {r}_{ρ} = \frac{ln ( {\vec{r}}_{ρ} / {\overleftarrow{r}}_{ρ} )}{{\vec{r}}_{ρ} - {\overleftarrow{r}}_{ρ}} = \frac{{A}_{ρ}}{{r}_{ρ}} \qquad [ 16 ]$$

where use has been made of Eq. [3]. Besides its appealing interpretation, this provides a linear relation between A_{ρ}, the dimensionless affinity, and r_{ρ}, the reaction rate, as in Ohm's law, albeit R_{ρ} changes with reaction conditions. There is, thus, now a complete correspondence between reaction networks and equivalent electrical circuits. Thus, not only Kirchhoff's laws are applicable, but resistances in series and parallel pathways can be treated just as in conventional electrical networks.
Reaction Network Analysis and Reduction (Steps 25 through 43)

The construction of a RR graph, even without a subsequent kinetic analysis, provides a powerful visualization tool and a deeper understanding of the reaction mechanism as compared to the traditional and computationally expensive analyses. A further use of the electrical circuit analogy and Kirchhoff's Laws provides a powerful new tool for a quantitative analysis and reduction of the RR network. Obviously, not all of the possible RRs are equally important. There are, in fact many alternate methods available for the reduction of kinetic systems (e.g., Gleiss, P. M.; Stadler, P. F.; Wagner, A.; Fell, D. A. Adv. Complex Systems 2001, 1, 1). We propose a different approach here based on the concept of reaction resistance in analogy to Ohm's law (step 25), followed by utilization of the techniques of electrical network analysis, to analyze and reduce RR networks (step 27). This turns out to be a particularly powerful method, allowing us to not only discriminate among the various RRs, but to also determine the slow, or rate-determining, as well as the quasi-equilibrium steps and, ultimately, when possible the derivation of a simple and accurate algebraic rate equation suitable for reactor analysis and design (step 43).

The definition of the linear reaction resistance, given by Eq. 16, is based on a linear constitutive relation in analogy with Ohm's law makes available a host of techniques used in the analysis of electrical circuits. However, it comes with the caveat that the reaction resistance so defined is not a constant, but depends upon the reaction conditions including the composition of the reaction mixture and especially the temperature, although less so than the coefficient \vec{r}_{ρ} in the conventional form of the De Donder equation. Thus, conclusions arrived at, e.g., regarding the slow steps, under one set of conditions may not be strictly applicable under a different set of conditions. Conceptually, of course, each elementary reaction step in a mechanism represents a different degree of “resistance” to the reaction progress. In fact, it is frequently assumed that all resistance resides in a single step, namely the rate-determining step (RDS), or sometimes in two rate-limiting steps.

It turns out that the large difference in the kinetics of elementary reactions, frequently many orders of magnitude, involved in a mechanism make RR networks eminently suitable for reduction. As mentioned above, it is quite likely that not all ORRs are equally significant. This means that branches corresponding to the insignificant ORRs may be dropped from the RR graph to simplify the overall kinetics and mechanism. Further, these considerations allow rationalization of the common assumptions in kinetics, for instance the RDS and pseudo-equilibrium assumptions.

The assumption in the following analysis is that the numerical results of a kinetic analysis under conditions of interest are available including concentrations (or surface coverages) of all intermediates, so that the reaction affinities and rates of all elementary reactions can be computed at step 25. The conclusions on reduction should eventually be checked under a different set of conditions to ensure that they are universally and not just locally applicable.

The overall procedure of simplification (steps 27 through 43) involves consideration of subgraphs or cycles of the complete RR graph. It is useful to begin with a fundamental RR matrix σ_{f} containing the RR with the smallest number of steps, the rest being the smallest ERRs. One may start with a comparison of the resistances of the branches in each of the empty routes or cycles. Each fundamental ERR may be divided into two parallel walks or paths between two given nodes (step 27), each path with the same affinity drop by virtue of KVL. Thus, the relative fluxes in the two pathways are equal to the ratio of the total resistances of the two paths,

$$\frac{{J}_{I}}{{J}_{II}} = \frac{{R}_{II}}{{R}_{I}} \qquad [ 17 ]$$

where the total resistance R_{I} or R_{II} of sequential branches between two nodes nᵢ and nⱼ is

$${R}_{k , {n}_{i} \to {n}_{j}} = {\sum }_{{n}_{i} \to {n}_{j}} {σ}_{k ρ}^{2} {R}_{ρ} \qquad [ 18 ]$$

and, of course, the reaction flux through the sequential branches

$${J}_{k} = \frac{{A}_{{n}_{i} -> {n}_{j}}}{{R}_{k , {n}_{i} \to {n}_{j}}} = \frac{{\sum }_{{}_{{n}_{i} \to {n}_{j}}} {σ}_{k ρ} {A}_{ρ}}{{R}_{k , {n}_{i} \to {n}_{j}}} \qquad [ 19 ]$$

For parallel paths between two nodes nᵢ and nⱼ, the total resistance is

$$\frac{1}{{R}_{Tot , {n}_{i} \to {n}_{j}}} = \frac{1}{{R}_{I , {n}_{i} \to {n}_{j}}} + \frac{1}{{R}_{II , {n}_{i} \to {n}_{j}}} + … \qquad [ 20 ]$$

If the resistance along one of the paths is much larger than that in the other (step 29), it would be safe to assume that the path contributes little to the OR rate and, hence, that path may be eliminated from the RR network (step 31). This, of course, concomitantly, simplifies the mechanism by pointing out the reaction steps that are kinetically inconsequential. As a corollary, if both pathways are significant, it implies that each of the parallel branches has a resistance, or slow steps, of comparable order as step 29. The procedure thus involves analyzing the resistances in all ERRs (and hence looping accordingly at step 41), and may result in a very significant simplification of the RR network. Recall that each link that is eliminated reduces by one the number of independent RRs.

The next step 35, 33 is to determine the relative resistances of the reaction steps in a given sequence. Since, at QSS, the rates of all reactions in a sequence is the same, this may be accomplished by a comparison of the affinity (step 33) drop over each of the branches in a sequence

$$\frac{{A}_{1}}{{A}_{2}} = \frac{{R}_{1}}{{R}_{2}} \qquad [ 21 ]$$

The order of series steps in a branch is unimportant from the point of view of this analysis, although it is usually significant mechanistically. The slowest steps are those with significant affinity drops, while those with affinity drops approaching zero may be considered to be at pseudo-equilibrium. This might allow development of explicit rate expressions for the OR. However, numerically, the rate of the OR may be alternately calculated from r_{OR}=A_{OR}/R_{OR}.

In the reaction resimulization of step 35, if the reaction rate is unchanged (decision step 33), then the RR network path with a higher resistance relative to the reaction mechanism is eliminated at step 37. If the reaction rate is changed (decision step 33), then the RR network path with high resistance is replaced at step 39.

Clearly, the above simplification and reduction steps 29, 31, 35, 37, 39 of FIG. 1 should provide a quantitative value for the tolerance that is appropriate for keeping or discarding various reaction steps. The problem may be simply formulated in terms of the overall resistance of the RR network. Thus, a reaction step may be neglected if the change in the overall resistance of the RR network does not exceed a chosen tolerance.

### CONCLUSION, RAMIFICATIONS, AND SCOPE

The determination of the chemical kinetics and mechanism for an OR based on a realistic set of elementary steps with predicted rate constants is an important current goal. Frequently, the OR is composed of sequential as well as parallel pathways and more than one significant ORR. All of these ORRs, in principle, affect the OR rate, since frequencies of all unit steps are finite. Furthermore, the slow steps and the significant ORRs may change with reaction conditions. However, the traditional approach has been to assume a RDS, all other steps being at quasi-equilibrium. Further, parallel pathways are rarely considered. More recently, the approach of microkinetics allows these assumptions to be abandoned. However, the numerical approach obscures an understanding of the key steps involved in the reaction mechanism and their interconnections.

What is proposed here is a new method and apparatus (approach) for the rigorous visualization and analysis of RR networks that is based on the combined use of graph theory, RR theory, microkinetic analysis, and an analogy with electrical circuits. The topological features of the RR graph are determined from an enumeration of RRs or simply from the application of the quasi-steady state (QSS) condition as discussed above. The approach is very different from that for reaction graphs used so far in the literature. In our approach, the reactions are represented by graph branches whereas the nodes simply represent the manner in which these reaction steps are hooked up to provide the various pathways followed by reactants in forming the products, whereas in the literature the nodes represent individual intermediate or terminal species. Our approach results in graphs that are also amenable to quantitative analysis based on the use of electrical circuit analogy and application of Kirchhoff's laws of current and potential adapted to reaction rate and affinity, respectively. Finally, making use of numerical microkinetic results, a systematic and rigorous analysis and simplification of the resulting mechanism and kinetics is performed (step 43). Such an understanding of significant elementary steps and RRs is crucial not only in developing practical rate expressions for performance analysis and design of reactors, but also form the basis of rational design of selective and active catalysts. Furthermore, the resulting RR graphs are amenable to depicting energy diagrams, with nodes representing unique values of thermodynamic state variables. Thus plausible energy diagrams are constructed at step 45 (FIG. 1) based on the resulting simplified RR graphs of step 43.

### EXAMPLES

1. Dihydrofolate Reductase (DHFR) System

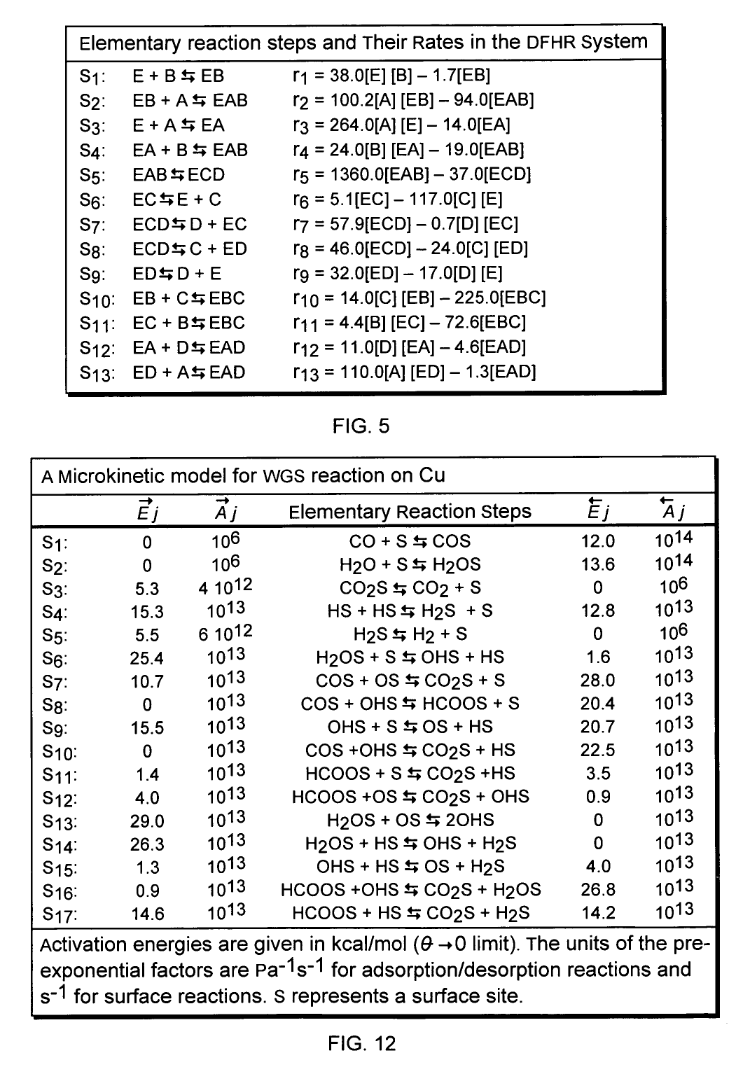

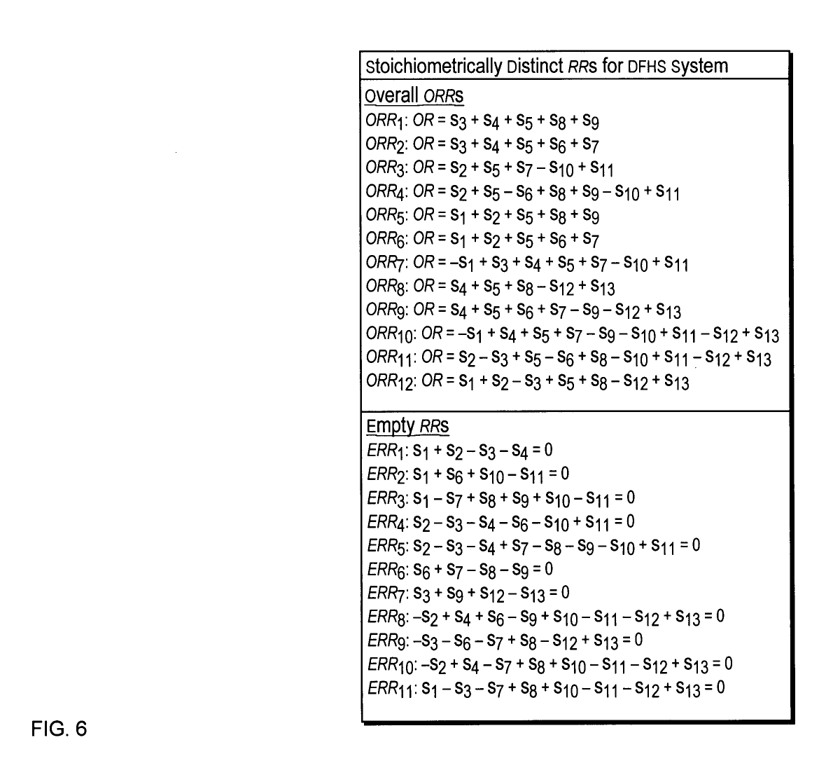

The mechanism describing the conversion of 7,8-dihydrofolate (A) and NADPH (B) into 5,6,7,8-tetrahydrofolate (C) and NADP⁺ (D) as catalyzed by the enzyme dihydrofolate reductase (E) along with a set of rate constants is presented in FIG. 5. The illustrated table is adapted from (Happel, J.; Otarod, M. J. Phys. Chem. B 2000, 104, 5209) This is a single OR of the type
A+B⇄C+D
involving multiple ORRs. The kinetics of this reaction have been intensively investigated both experimentally (Andrews, J.; Fierke, C. A.; Birdsall, B.; Ostler, G.; Feeney, J.; Roberts, G. C. K.; Benkovic, S. J. Biochemistry 1989, 28; Fierke, C. A.; Benkovic, S. J. Biochemistry 1989, 28, 5478; Fierke, C. A.; Johnson, K. A.; Benkovic, S. J. Biochemistry 1987, 26, 4085; Thillet, J.; Adams, J. A.; Benkovic, S. J. Biochemistry 1990, 5195) and theoretically, employing the theory of direct RRs and the conventional King-Altman formalism (Chen, T.-S.; Chern, J.-M. Chem. Eng. Sci. 2003, 58, 1407; King, E. L.; Altman, C. J. Phys. Chem. 1956, 60, 6676; Poland, D. J. Phys. Chem. 1989, 93, 3613). The enumeration of direct RRs in this system has been considered by Happel and Sellers (Happel, J.; Otarod, M. J. Phys. Chem. B 2000, 104, 5209; Happel, J.; Sellers, P. H. J. Phys. Chem. 1992, 96, 2593; Happel, J.; Sellers, P. H. J. Phys. Chem. 1995, 99, 6595). Their results are presented in FIG. 6.

The rate constants of the elementary reaction steps used in previous publications are not thermodynamically consistent, i.e., Kirchhoffs Voltage Law (KVL) is not followed as is rendered transparent by the current approach. In other words, the affinity of the overall reaction expressed via the affinities of the elementary reaction steps takes on slightly different values for different ORRs. Alternatively, the affinities of the elementary reaction steps comprising ERR do not sum to zero. For instance, from FIG. 6, for ER₂, i.e.,
s₁+s₆+s₁₀−s₁₁=0
the affinities follow
A₁+A₆+A₁₀−A₁₁=0.
In turn, the rate constants of these elementary reaction steps are subject to the following constraint

$$( \frac{{{k}^{->}}_{1}}{{\overleftarrow{k}}_{1}} ) ( \frac{{{k}^{->}}_{6}}{{\overleftarrow{k}}_{6}} ) ( \frac{{{k}^{->}}_{10}}{{\overleftarrow{k}}_{10}} ) {( \frac{{{k}^{->}}_{11}}{{\overleftarrow{k}}_{11}} )}^{- 1} = 1$$

Using the data from FIG. 5, it can be readily verified that this product is equal to 1.378 rather than 1. To eliminate this thermodynamic inconsistency, we have modified the rate constant in the reverse direction for reaction s₁₁, i.e., the original value [symbol] ₁₁=100 has been replaced with [symbol] ₁₁=72.57. Other reaction constants that have been modified are \vec{k}₂, \vec{k}₇, and \vec{k}₁₂. It should be noted that these modifications do not affect the overall kinetics of the process.

For the purpose of this illustration, all of the numerical simulations described below for this system were performed for a batch reactor under the following set of initial conditions: [A]=[B]=0.9, [C]=[D]=0.1 and [E]=1, while [EA]=[EB]=[AC]=[ES]=[EAB]=[EBC]=[ECD]=[EAD]=0, in arbitrary units. The numerical results were used to compute reaction step resistances and affinities.

The system comprises q=8 independent intermediates (EA, EB, EC, ED, EAB, ECD, EBC and EAD) and n=4 terminal species (A, B, C and D). Our starting point in the construction of the RR graph is the overall stoichiometric matrix

$$EA \qquad EB \qquad EC \qquad ED \qquad EAB \qquad EBC \qquad ECD \qquad EAD \qquad A \qquad B \qquad C \qquad D$$

$$v = [ \begin{matrix}0 & 0 & 0 & 0 & 0 & 0 & 0 & 0 & - 1 & - 1 & + 1 & + 1 \\ 0 & + 1 & 0 & 0 & 0 & 0 & 0 & 0 & 0 & - 1 & 0 & 0 \\ 0 & - 1 & 0 & 0 & + 1 & 0 & 0 & 0 & - 1 & 0 & 0 & 0 \\ + 1 & 0 & 0 & 0 & 0 & 0 & 0 & 0 & - 1 & 0 & 0 & 0 \\ - 1 & 0 & 0 & 0 & + 1 & 0 & 0 & 0 & 0 & - 1 & 0 & 0 \\ 0 & 0 & 0 & 0 & - 1 & 0 & + 1 & 0 & 0 & 0 & 0 & 0 \\ 0 & 0 & - 1 & 0 & 0 & 0 & 0 & 0 & 0 & 0 & + 1 & 0 \\ 0 & 0 & + 1 & 0 & 0 & 0 & - 1 & 0 & 0 & 0 & 0 & + 1 \\ 0 & 0 & 0 & + 1 & 0 & 0 & - 1 & 0 & 0 & 0 & + 1 & 0 \\ 0 & 0 & 0 & - 1 & 0 & 0 & 0 & 0 & 0 & 0 & 0 & + 1 \\ 0 & - 1 & 0 & 0 & 0 & + 1 & 0 & 0 & 0 & 0 & - 1 & 0 \\ 0 & 0 & - 1 & 0 & 0 & + 1 & 0 & 0 & 0 & - 1 & 0 & 0 \\ - 1 & 0 & 0 & 0 & 0 & 0 & 0 & + 1 & 0 & 0 & 0 & - 1 \\ 0 & 0 & 0 & - 1 & 0 & 0 & 0 & + 1 & - 1 & 0 & 0 & 0\end{matrix} ] \begin{matrix}OR \\ {s}_{1} \\ {s}_{2} \\ {s}_{3} \\ {s}_{4} \\ {s}_{5} \\ {s}_{6} \\ {s}_{7} \\ {s}_{8} \\ {s}_{9} \\ {s}_{10} \\ {s}_{11} \\ {s}_{12} \\ {s}_{13}\end{matrix}$$

Because the concentrations of the intermediates are subject to an enzyme balance, E is eliminated from the above consideration:
[E]+[EA]+[EB]+[EC]+[ED]+[EAB]+[ECD]+[EBC]+[EAD]=[E₀]
where [E₀] is the total concentration of enzyme. Therefore, since q=rankα=8, the number of nodes in the RR graph is equal to N=q+2=8+2=10. The incidence matrix M of the RR graph may be obtained by performing row operations on the transposed overall stoichiometric matrix ν^{T}. After a series of elementary row operations such that each column has no more than a +1, and a −1, the balance of elements being zero, we obtain.

$$\begin{matrix} OR & {s}_{1} & {s}_{2} & {s}_{3} & {s}_{4} & {s}_{5} & {s}_{6} & {s}_{7} & & {s}_{8} & {s}_{9} & & {s}_{10} & {s}_{11} & {s}_{12} & {s}_{13}\end{matrix}$$

$$M = [ \begin{matrix}+ 1 & 0 & + 1 & 0 & + 1 & 0 & 0 & 0 & 0 & 0 & 0 & 0 & 0 & 0 \\ 0 & + 1 & - 1 & 0 & 0 & 0 & 0 & 0 & 0 & 0 & - 1 & 0 & 0 & 0 \\ 0 & 0 & 0 & + 1 & - 1 & 0 & 0 & 0 & 0 & 0 & 0 & 0 & - 1 & 0 \\ 0 & - 1 & 0 & - 1 & 0 & 0 & + 1 & 0 & 0 & + 1 & 0 & 0 & 0 & 0 \\ 0 & 0 & 0 & 0 & 0 & 0 & 0 & 0 & 0 & 0 & + 1 & + 1 & 0 & 0 \\ 0 & 0 & 0 & 0 & 0 & 0 & 0 & 0 & 0 & 0 & 0 & 0 & + 1 & + 1 \\ 0 & 0 & 0 & 0 & 0 & 0 & - 1 & + 1 & 0 & 0 & 0 & - 1 & 0 & 0 \\ 0 & 0 & 0 & 0 & 0 & 0 & 0 & 0 & + 1 & - 1 & 0 & 0 & 0 & - 1 \\ 0 & 0 & 0 & 0 & 0 & + 1 & 0 & - 1 & - 1 & 0 & 0 & 0 & 0 & 0 \\ - 1 & 0 & 0 & 0 & 0 & - 1 & 0 & 0 & 0 & 0 & 0 & 0 & 0 & 0\end{matrix} ] \begin{matrix}{n}_{1} \\ {n}_{2} \\ {n}_{3} \\ {n}_{4} \\ {n}_{5} \\ {n}_{6} \\ {n}_{7} \\ {n}_{8} \\ {n}_{9} \\ {n}_{10}\end{matrix}$$

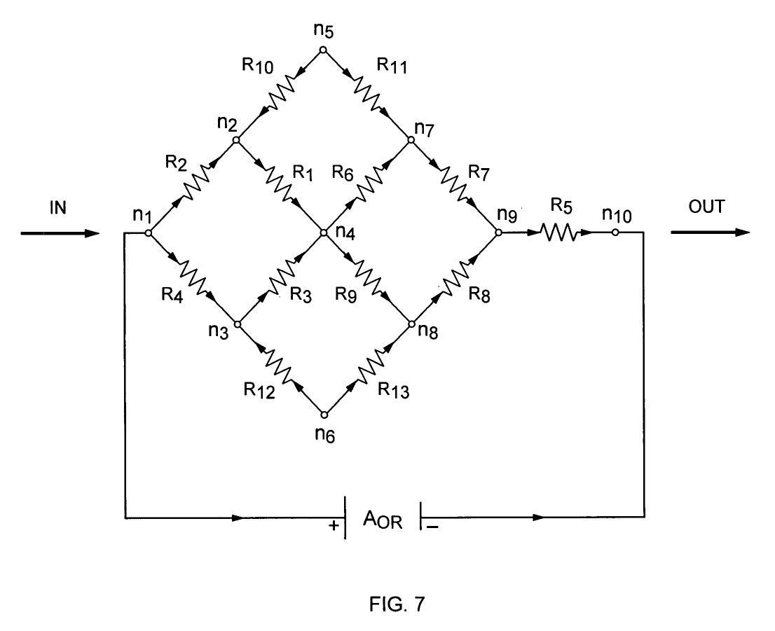

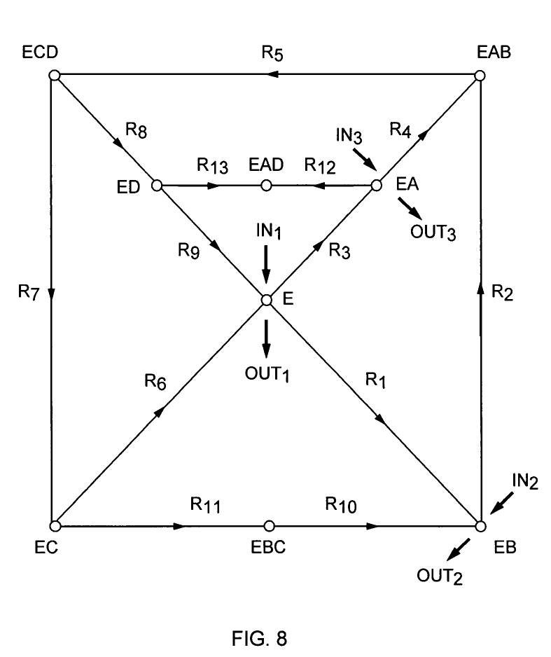

The RR graph corresponding to this kinetic mechanism is easily drawn and is shown in FIG. 7. It is evident that the reaction graph incorporates the complete list of both ORRs and ERRs listed in FIG. 6. For comparison, we present, in FIG. 8, the conventional reaction graph, i.e., a graph in which the nodes represent the intermediates, since it is possible to do so in this linear kinetic mechanism. It can be seen that the conventional graph is not amenable to RR analysis. Thus, in order to get a complete set of direct RRs, one needs to employ three different starting and ending points on the graph.

Alternatively, the RR graph may be constructed from an arbitrarily selected set of linearly independent RRs, chosen from FIG. 6. According to the Horiuti-Temkin theorem, the number of linearly independent RRs for this system is equal to L=p−rank α=13−8=5. These 5 independent RRs may be arbitrarily selected from the list of direct RRs in FIG. 6. For example, a set of 5 linearly independent RRs is
s₁+s₂−s₃−s₄=0 (ERRI) RR_{I}:
s₃+s₄+s₅+s₆+s₇=OR (ORR₂) RR_{II}:
s₃+s₄+s₅+s₈+s₉=OR (RR₁) RR_{III}:
−s₁+s₃+s₄+s₅+s₇−s₁₀+s₁₁=OR (ORR₇) RR_{IV}:
s₄+s₅+s₈−s₁₂+s₁₃=OR (ORR₈) RR_{V}:

As can be seen, the first RR from this set is an ERR. Indeed

| | | σⱼ |
|---|---|---|
| s₁: | E + B [symbol] EB | 1 |
| s₂: | EB + A [symbol] EAB | 1 |
| s₃: | E + A [symbol] EA | −1 |
| s₄: | EA + B [symbol] EAB | −1 |
| Net: | 0 [symbol] 0 | |

The next 4 RRs, however, are ORRs. For instance, the second RR is

| | | σⱼ |
|---|---|---|
| s₃: | E + A [symbol] EA | 1 |
| s₄: | EA + B [symbol] EAB | 1 |
| s₅: | EAB [symbol] ECD | 1 |
| s₆: | EC [symbol] E + C | 1 |
| s₇: | ECD [symbol] D + EC | 1 |
| Net: | A + B [symbol] C + D | |

It is seen that, after reordering the elementary reaction steps, the fundamental RR matrix may be presented in the form

$${\overbrace{\begin{matrix}OR & {s}_{1} & {s}_{3} & {s}_{4} & {s}_{5} & {s}_{7} & {s}_{8} & {s}_{10} {s}_{12}\end{matrix}}}^{twigs} { \overbrace{\begin{matrix}{s}_{2} & {s}_{6} & {s}_{9} & {s}_{11} {s}_{13}\end{matrix}}}^{links}$$

$${σ}_{f} = [ \begin{matrix}0 & + 1 & - 1 & - 1 & 0 & 0 & 0 & 0 & 0 & ⋮ & + 1 & 0 & 0 & 0 & 0 \\ - 1 & 0 & + 1 & + 1 & + 1 & + 1 & 0 & 0 & 0 & ⋮ & 0 & + 1 & 0 & 0 & 0 \\ - 1 & 0 & + 1 & + 1 & + 1 & 0 & + 1 & 0 & 0 & ⋮ & 0 & 0 & + 1 & 0 & 0 \\ - 1 & - 1 & + 1 & + 1 & + 1 & + 1 & 0 & - 1 & 0 & ⋮ & 0 & 0 & 0 & + 1 & 0 \\ - 1 & 0 & 0 & + 1 & + 1 & 0 & + 1 & 0 & - 1 & ⋮ & 0 & 0 & 0 & 0 & + 1\end{matrix} ] \begin{matrix}{RR}_{I} \\ {RR}_{II} \\ {RR}_{III} \\ {RR}_{IV} \\ {RR}_{V}\end{matrix}$$

This results in the following fundamental cut-set matrix

$${\overbrace{\begin{matrix}OR & {s}_{1} & {s}_{3} & {s}_{4} & {s}_{5} & {s}_{7} & {s}_{8} & {s}_{10} {s}_{12}\end{matrix}}}^{twigs} { \overbrace{\begin{matrix}{s}_{2} & {s}_{6} & {s}_{9} & {s}_{11} {s}_{13}\end{matrix}}}^{links}$$

$${X}_{f} = [ \begin{matrix}+ 1 & 0 & 0 & 0 & 0 & 0 & 0 & 0 & 0 & ⋮ & 0 & + 1 & + 1 & + 1 & + 1 \\ 0 & + 1 & 0 & 0 & 0 & 0 & 0 & 0 & 0 & ⋮ & - 1 & 0 & 0 & + 1 & 0 \\ 0 & 0 & + 1 & 0 & 0 & 0 & 0 & 0 & 0 & ⋮ & + 1 & - 1 & - 1 & - 1 & 0 \\ 0 & 0 & 0 & + 1 & 0 & 0 & 0 & 0 & 0 & ⋮ & + 1 & - 1 & - 1 & - 1 & - 1 \\ 0 & 0 & 0 & 0 & + 1 & 0 & 0 & 0 & 0 & ⋮ & 0 & - 1 & - 1 & - 1 & - 1 \\ 0 & 0 & 0 & 0 & 0 & + 1 & 0 & 0 & 0 & ⋮ & 0 & - 1 & 0 & - 1 & 0 \\ 0 & 0 & 0 & 0 & 0 & 0 & + 1 & 0 & 0 & ⋮ & 0 & 0 & - 1 & 0 & - 1 \\ 0 & 0 & 0 & 0 & 0 & 0 & 0 & + 1 & 0 & ⋮ & 0 & 0 & 0 & + 1 & 0 \\ 0 & 0 & 0 & 0 & 0 & 0 & 0 & 0 & + 1 & ⋮ & 0 & 0 & 0 & 0 & + 1\end{matrix} ]$$

The incidence matrix M may next be readily obtained by elementary row operations on X_{f} followed by the addition of one more row.

Kirchhoff's Voltage Law (KVL) and Current Law (KCL) may be written as A^{(l)}=−σ₁A^{(t)} and r^{(t)}=σ₁^{T}J, respectively. By the convention r₀=−r_{OR}, along with r^{(l)}=J, the following relationships for the link affinities in terms of the twig affinities, and the twig rates in terms of the link rates are derived

KVL
A₁+A₂−A₃−A₄=0
A_{OR}=A₃+A₄+A₅+A₆+A₁₀
A_{OR}=A₃+A₄+A₅+A₈+A₉
A_{OR}=A₁+A₃+A₄+A₅−A₁₀+A₁₁
A_{OR}=A₄+A₅+A₈−A₁₂+A₁₃

KCL

| r^{(t)} = σₜ^{T} J | r^{(l)} = J |
|---|---|
| r_{OR} = J_{II} + J_{III} + J_{IV} + J_{V} | r₂ = J_{I} |
| r₁ = J_{I} − J_{IV} | r₆ = J_{II} |
| r₃ = −J_{I} + J_{II} + J_{III} + J_{IV} | r₉ = J_{III} |
| r₄ = −J_{I} + J_{II} + J_{III} + J_{IV} | r₁₁ = J_{IV} |
| r₅ = J_{II} + J_{III} + J_{IV} + J_{V} | r₁₃ = J_{V} |
| r₇ = J_{II} + J_{IV} | |
| r₈ = J_{III} + J_{V} | |
| r₁₀ = −J_{IV} | |
| r₁₂ = −J_{V} | |

It should be noted that the first KVL equation is representative of an ERR, and hence, the sum of the affinities involved equates to zero.

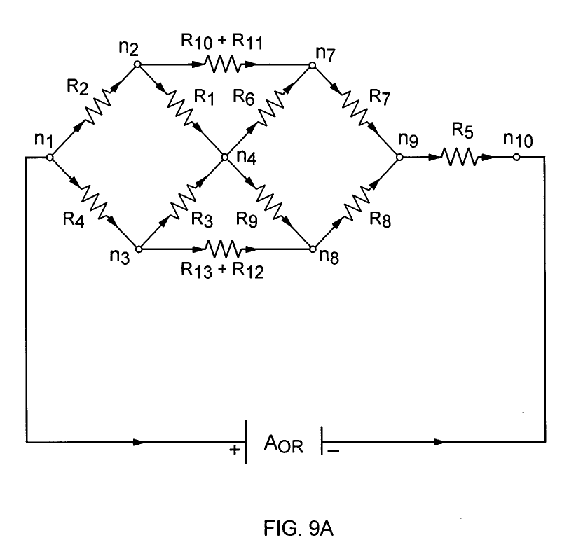

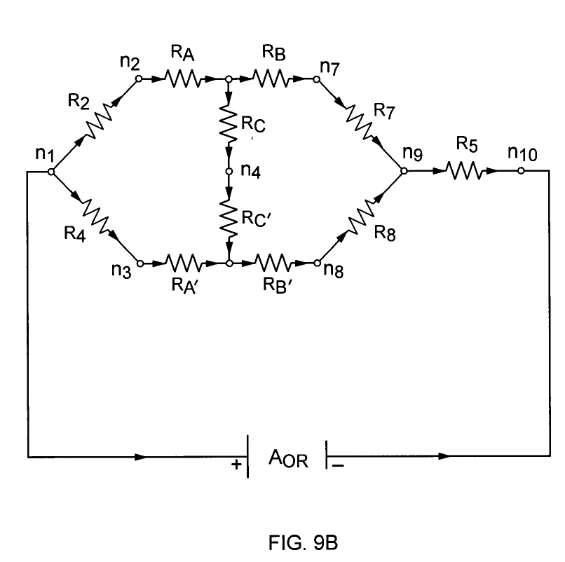

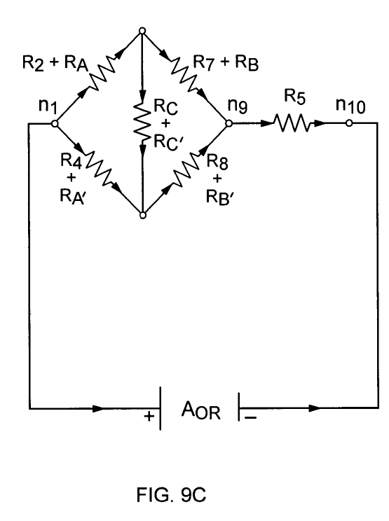

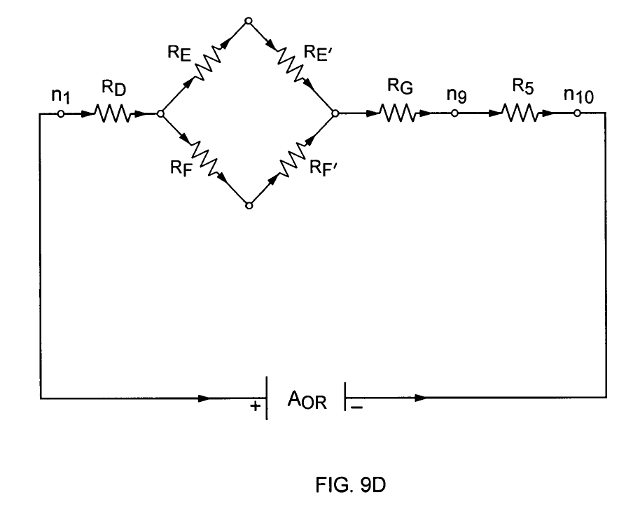

In turn, the overall resistance may be evaluated from the individual resistances of the elementary reaction steps using the conventional electrical circuit theory. The procedure, involving a series of Δ-Y conversions, is illustrated in FIG. 9 and comprises the following steps. First, we add the resistances that are connected in series, i.e., R₁₀+R₁₁, R₁₂+R₁₃ and rearrange the graph as shown in FIG. 9A. Second, we apply a set of Δ-Y conversions thus arriving at FIG. 9B. Third, after again combining the resistances in series, a second set of Δ-Y conversions is applied and the graph is rearranged according to FIG. 9C. A final set of Δ-Y conversions results in a simple network (FIG. 9D) which yields the following expression for the overall resistance

$$\begin{matrix}{R}_{OR} = {R}_{D} + {R}_{G} + {R}_{5} + ( \frac{1}{{R}_{E} + {R}_{{E}^{′}}} + \frac{1}{{R}_{F} + {R}_{{F}^{′}}} ) where \\ {R}_{A} = \frac{{R}_{1} ( {R}_{10} + {R}_{11} )}{{R}_{1} + {R}_{6} + ( {R}_{10} + {R}_{11} )} \\ {R}_{B} = \frac{{R}_{6} ( {R}_{10} + {R}_{11} )}{{R}_{1} + {R}_{6} + ( {R}_{10} + {R}_{11} )} \\ {R}_{C} = \frac{{R}_{1} {R}_{6}}{{R}_{1} + {R}_{6} + ( {R}_{10} + {R}_{11} )} \\ {R}_{{A}^{′}} = \frac{{R}_{3} ( {R}_{12} + {R}_{13} )}{{R}_{3} + {R}_{9} + ( {R}_{12} + {R}_{13} )} \\ {R}_{{B}^{′}} = \frac{{R}_{9} ( {R}_{12} + {R}_{13} )}{{R}_{3} + {R}_{9} + ( {R}_{12} + {R}_{13} )} \\ {R}_{{C}^{′}} = \frac{{R}_{3} {R}_{9}}{{R}_{3} + {R}_{9} + ( {R}_{12} + {R}_{13} )} \\ {R}_{D} = \frac{( {R}_{2} + {R}_{A} ) ( {R}_{4} + {R}_{{A}^{′}} )}{( {R}_{2} + {R}_{A} ) + ( {R}_{4} + {R}_{{A}^{′}} ) + ( {R}_{C} + {R}_{{C}^{′}} )} \\ {R}_{E} = \frac{( {R}_{2} + {R}_{A} ) ( {R}_{C} + {R}_{{C}^{′}} )}{( {R}_{2} + {R}_{A} ) + ( {R}_{4} + {R}_{{A}^{′}} ) + ( {R}_{C} + {R}_{{C}^{′}} )} \\ {R}_{F} = \frac{( {R}_{4} + {R}_{{A}^{′}} ) ( {R}_{C} + {R}_{{C}^{′}} )}{( {R}_{2} + {R}_{A} ) + ( {R}_{4} + {R}_{{A}^{′}} ) + ( {R}_{C} + {R}_{{C}^{′}} )} \\ {R}_{{E}^{′}} = \frac{( {R}_{7} + {R}_{B} ) ( {R}_{C} + {R}_{{C}^{′}} )}{( {R}_{7} + {R}_{B} ) + ( {R}_{8} + {R}_{{B}^{′}} ) + ( {R}_{C} + {R}_{{C}^{′}} )} \\ {R}_{{F}^{′}} = \frac{( {R}_{8} + {R}_{{B}^{′}} ) ( {R}_{C} + {R}_{{C}^{′}} )}{( {R}_{7} + {R}_{B} ) + ( {R}_{8} + {R}_{{B}^{′}} ) + ( {R}_{C} + {R}_{{C}^{′}} )} \\ {R}_{G} = \frac{( {R}_{7} + {R}_{B} ) ( {R}_{8} + {R}_{{B}^{′}} )}{( {R}_{7} + {R}_{B} ) + ( {R}_{8} + {R}_{{B}^{′}} ) + ( {R}_{C} + {R}_{{C}^{′}} )}\end{matrix} \qquad [ 22 ]$$

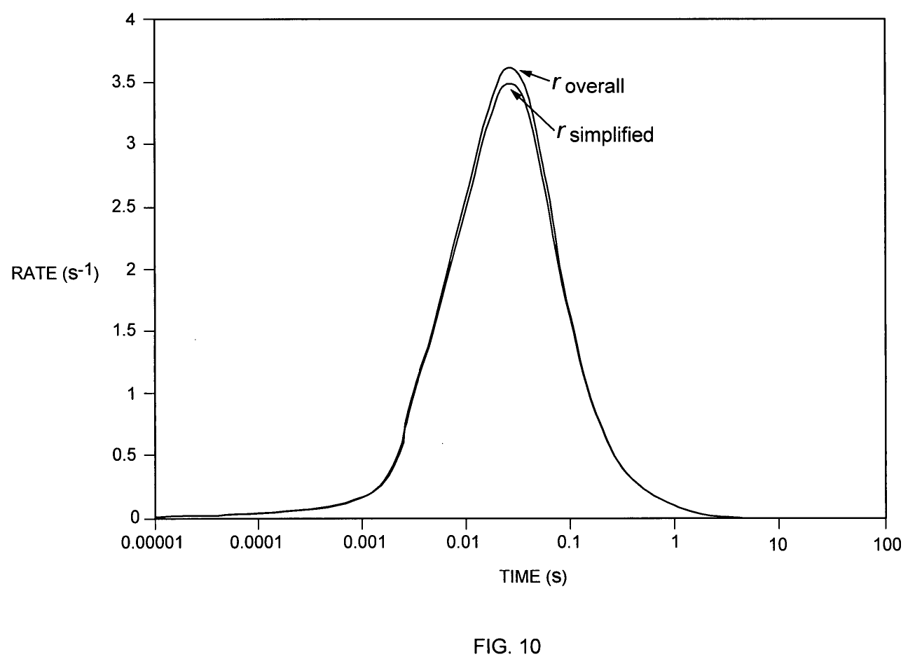

The overall rate as a function of time in a batch reactor for the conditions mentioned above is presented in FIG. 10.

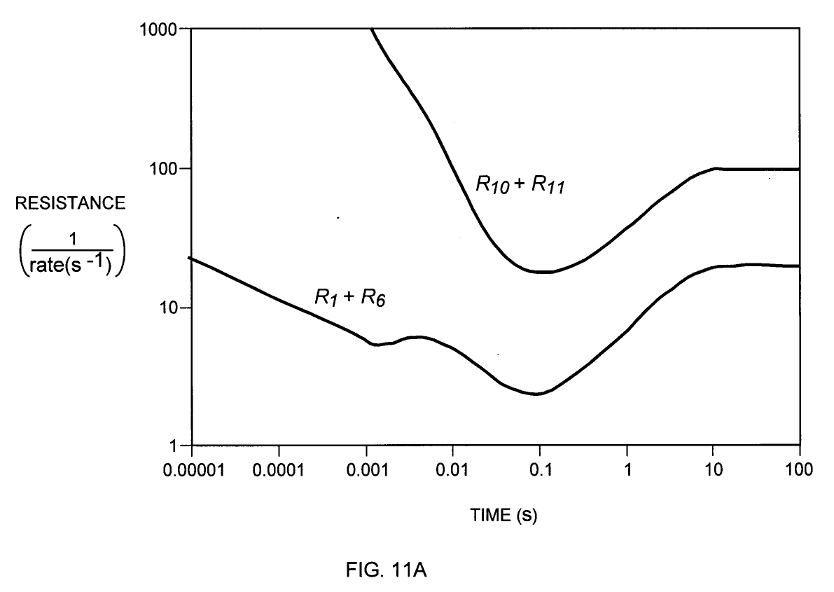

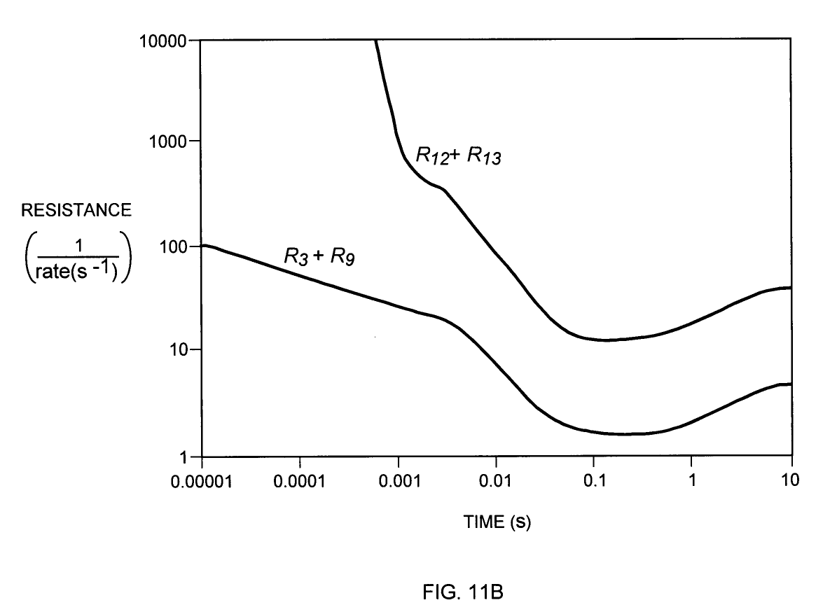

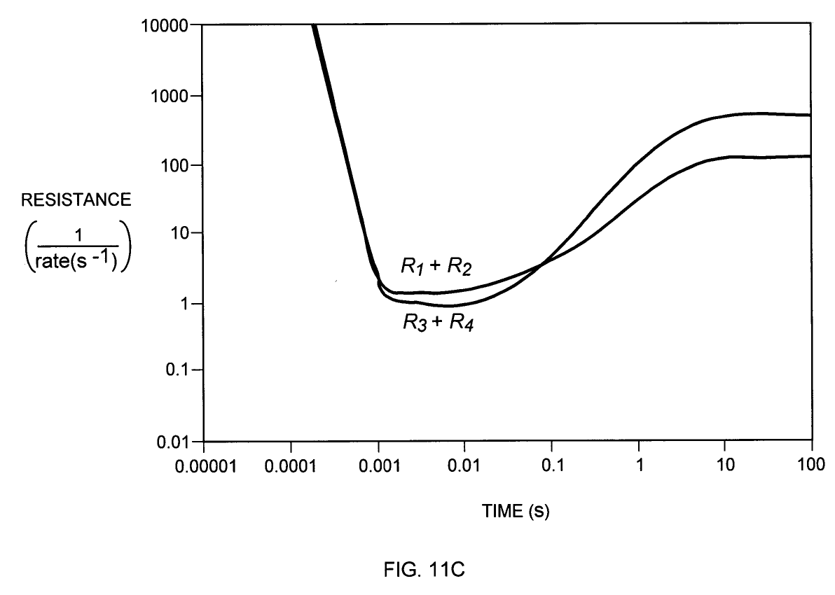

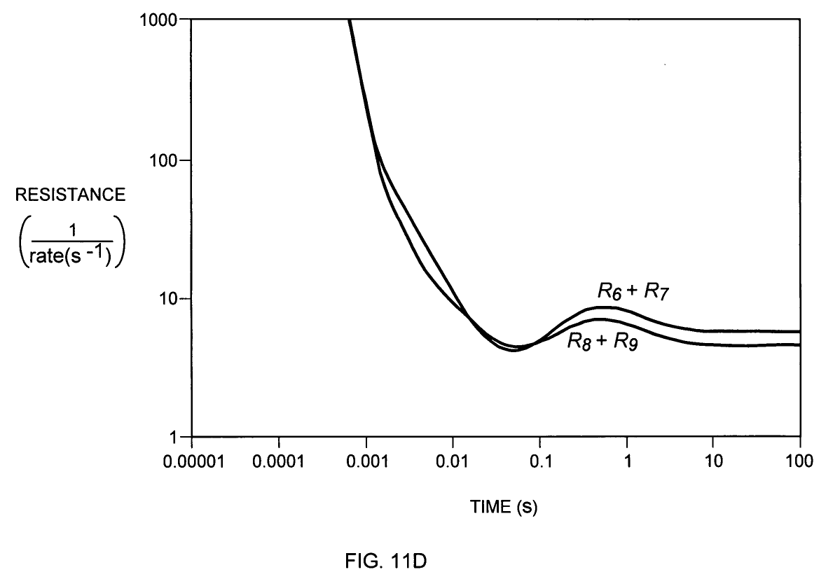

Next, we analyze the RR graph with an eye for simplification and reduction. This can be achieved by evaluating separately the resistances along parallel pathways between two nodes. In FIG. 11A and FIG. 11B, we compare the pathways between nodes n₂ and n₇, and n₃ and n₈, respectively. It is seen that there are sufficient differences in the resistances to justify neglecting the pathways consisting of R₁₀+R₁₁, and R₁₂+R₁₃ (FIG. 9A). Concomitantly, neglecting these pathways does not affect the overall resistance of the RR network. FIG. 11C and FIG. 11D provide a similar comparison between the pathways involving nodes n₁ and n₄, and n₄ and n₉. Here, however, the resistances are sufficiently close that no further reduction may be performed. We thus conclude that the elementary reaction steps s₁₀, s₁₁, s₁₂ and s₁₃ may be dropped from the mechanism. As a result, the overall resistance of the reaction network is substantially simplified

$${R}_{OR} = {( \frac{1}{{R}_{1} + {R}_{2}} + \frac{1}{{R}_{3} + {R}_{4}} )}^{- 1} + {( \frac{1}{{R}_{6} + {R}_{7}} + \frac{1}{{R}_{8} + {R}_{9}} )}^{- 1} + {R}_{5} \qquad [ 23 ]$$

As can be seen from FIG. 10, the simplified overall rate is in good agreement with the overall rate of the complete network.
2. Water-Gas Shift Reaction

A 17-step microkinetic model for the WGSR on Cu(111) is presented in FIG. 12, where S denotes a catalyst surface site. This model consists of adsorption (s₁ and s₂) and desorption (s₃ and s₅) steps, along with several surface reactions. The energetics were predicted a priori using the Unity Bond Index-Quadratic Exponential Potential (UBI-QEP) method (Shustorovich, E.; Sellers, H. Surf Sci. 1998, 31, 1) for the activation energies, and the transition-state theory, assuming an immobile transition state without rotation, for the pre-exponential factors, adjusted slightly to conform to the overall thermodynamics of the WGS reaction.

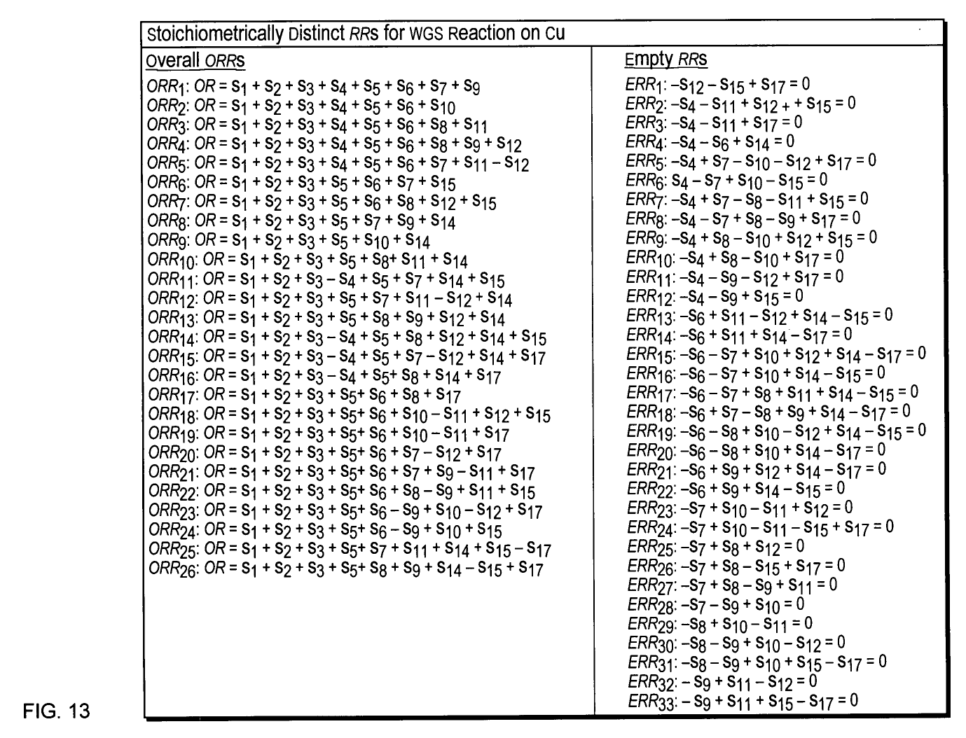

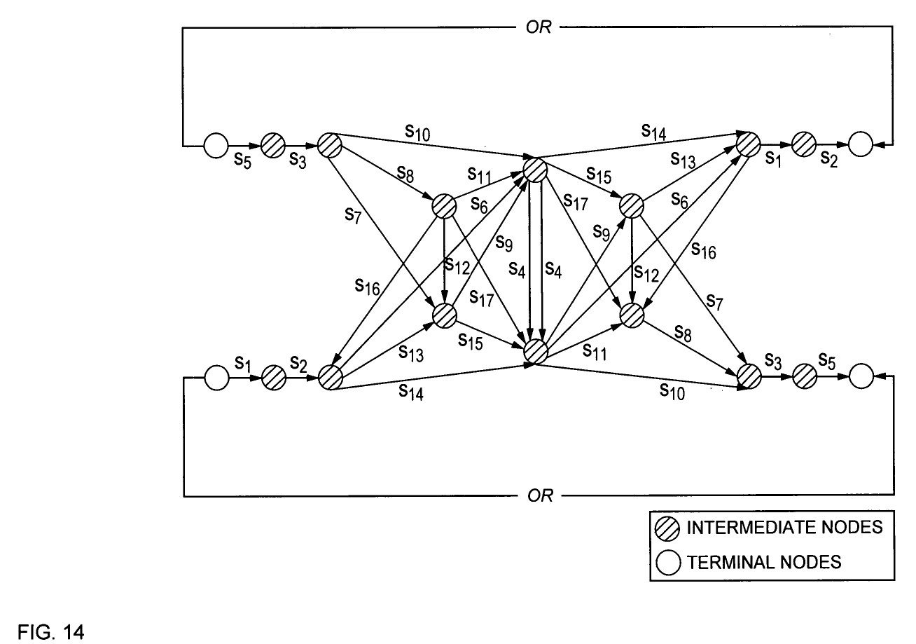

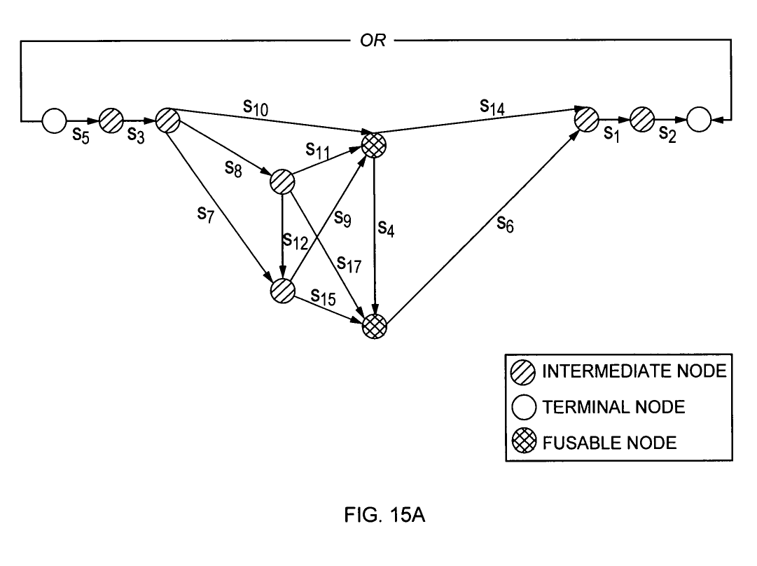

Using the stoichiometric algorithm described above, the complete list of ORRs, ERRs, INs, and TNs, were enumerated. A partial list is given in FIG. 13. It became evident from a perusal of the list that this is a non-minimal mechanism as there were a number of ORRs with a σ_{gρ}=±2. Thus, the non-minimal RR graph for WGS reaction was constructed following the algorithm described above, and the results are depicted in FIG. 14. This symmetrical graph has all of the RRs enumerated as trails and shows how the various mechanistic steps are hooked up.

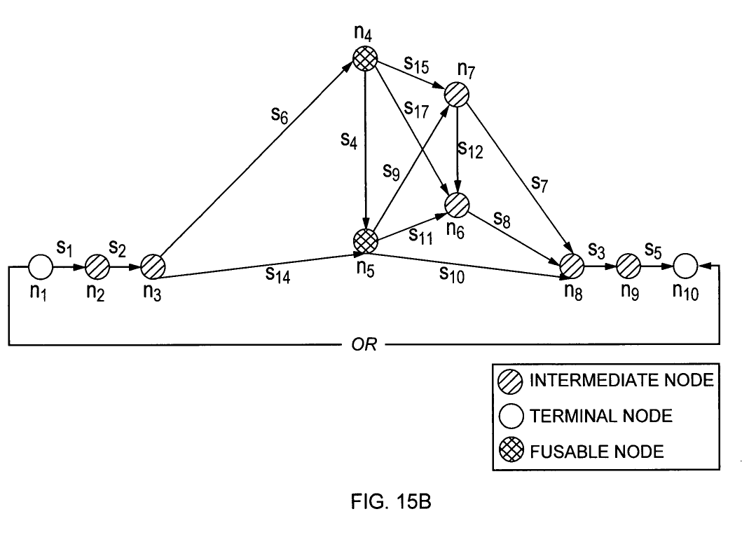

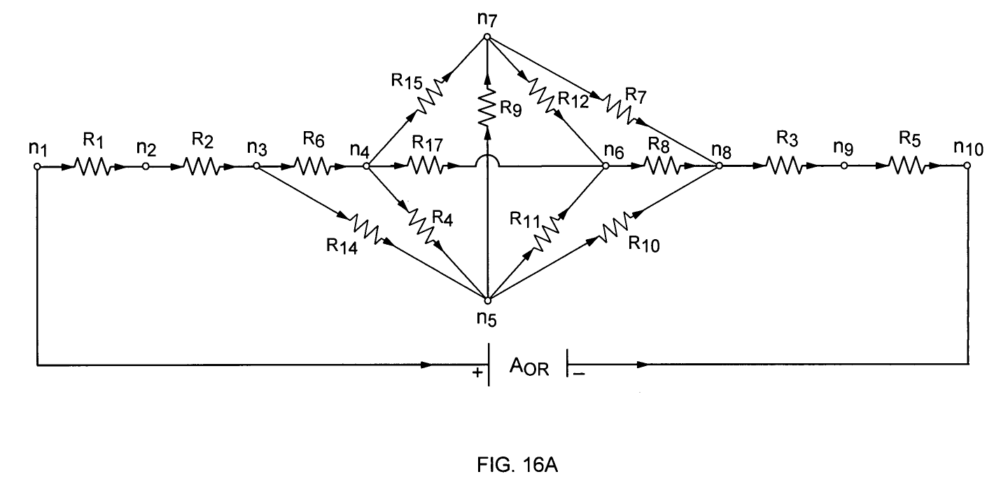

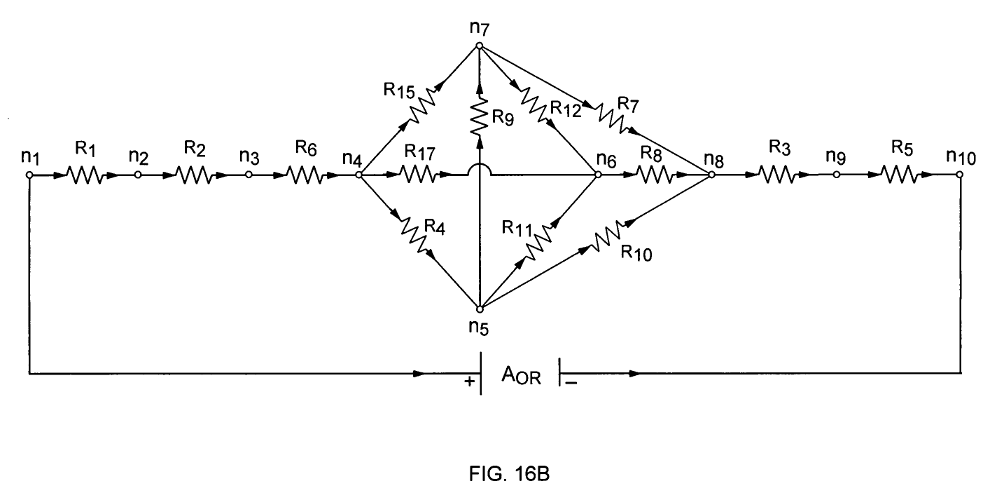

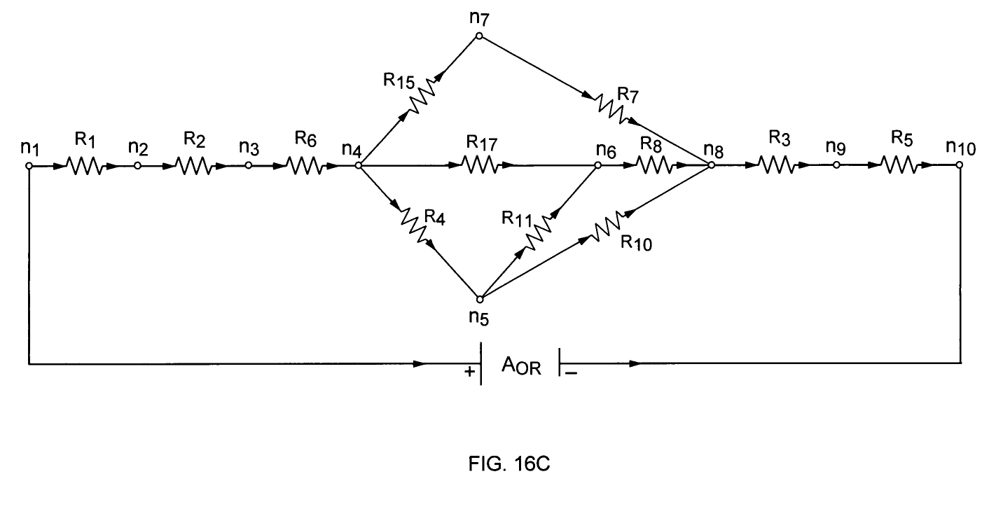

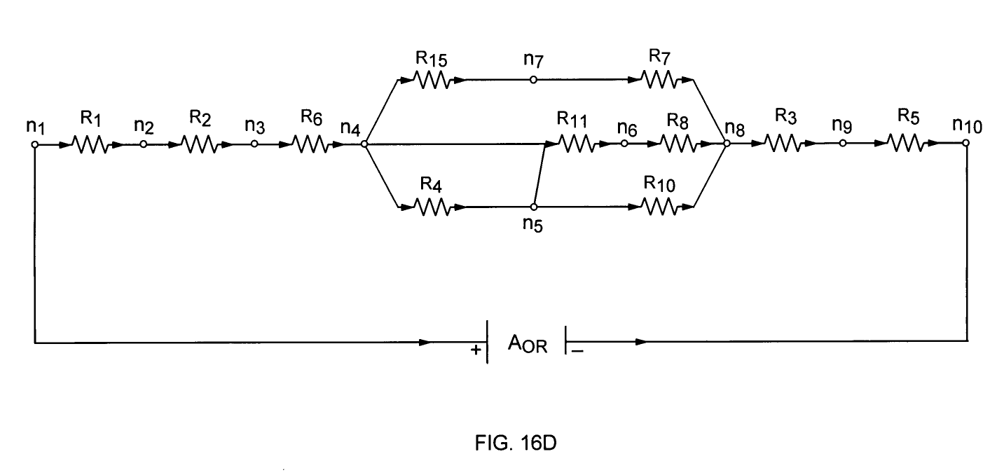

In investigating the cause of non-minimality, it was found that if two of the elementary reaction steps, namely, s₁₃ and s₁₆, were removed from the complete kinetic model, only unit stoichiometric numbers resulted. Furthermore, a microkinetic analysis showed that both s₁₃ and s₁₆ are not major contributors to the kinetics of the WGSR, since neglecting these had no effect on the accuracy of the predictions. Therefore, we eliminated these steps from FIG. 14, which resulted in two equivalent disconnected minimal subgraphs after the fused nodes were separated as shown in FIG. 15, either of which could be used for further analysis. Thus, FIG. 15B was converted into an equivalent electrical circuit as shown in FIG. 16A, by replacing the lines denoting mechanistic steps by resistances R_{ρ} and the OR by a voltage source.

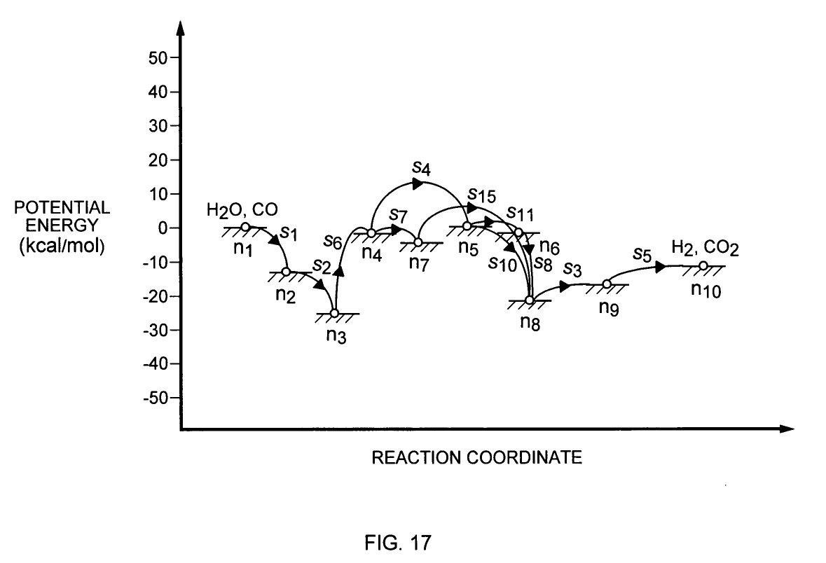

Using Eq. [16], the resistance of each of the steps was calculated and compared at various temperatures in order to further simplify stepwise the minimal graph, as shown in FIG. 16. The illustrated simplifications, thus, leave us with a reduced RR network comprising of 11 elementary reaction steps and only three parallel pathways (FIG. 16D). The fact that nodes represent unique thermodynamic potential functions can also be used to easily generate a reaction energy diagram, as shown in FIG. 17, for the three dominant pathways.

In fact, a further comparison of the sequential resistances in the three parallel pathways reveals the bottleneck steps (RDS) in each of the three parallel routes. Thus, the remaining steps can be treated as being at quasi-equilibrium. Then, following the conventional LHHW formalism, an explicit rate expression for the OR may be obtained:

$${r}_{OR} = \frac{{{k}^{->}}_{6} {K}_{2} {p}_{{H}_{2} O} {θ}_{0}^{2} [ ( {{k}^{->}}_{8} + {{k}^{->}}_{10} ) {K}_{1} {p}_{CO} + {{k}^{->}}_{15} \frac{{p}_{{H}_{2}}^{1 / 2}}{{( {K}_{4} {K}_{5} )}^{1 / 2}} ( \frac{{{k}^{->}}_{7} {K}_{2} {K}_{5} {K}_{15} {p}_{CO}}{{{k}^{->}}_{7} {K}_{2} {K}_{5} {K}_{15} {p}_{CO} + {{k}^{->}}_{15} {p}_{{H}_{2}}} ) ]}{[ \frac{{{k}^{->}}_{6}}{{K}_{6}} + {{k}^{->}}_{15} ( \frac{{{k}^{->}}_{7} {K}_{2} {K}_{5} {K}_{15} {p}_{CO}}{{{k}^{->}}_{7} {K}_{2} {K}_{5} {K}_{15} {p}_{CO} + {{k}^{->}}_{15} {p}_{{H}_{2}}} ) ] \frac{{p}_{{H}_{2}}^{1 / 2}}{{( {K}_{4} {K}_{5} )}^{1 / 2}} + ( {{k}^{->}}_{8} + {{k}^{->}}_{10} ) {K}_{1} {p}_{CO}} ( 1 - \frac{{p}_{{CO}_{2}} {p}_{{H}_{2}}}{{Kp}_{{H}_{2} O} {p}_{CO}} ) \qquad [ 24 ]$$

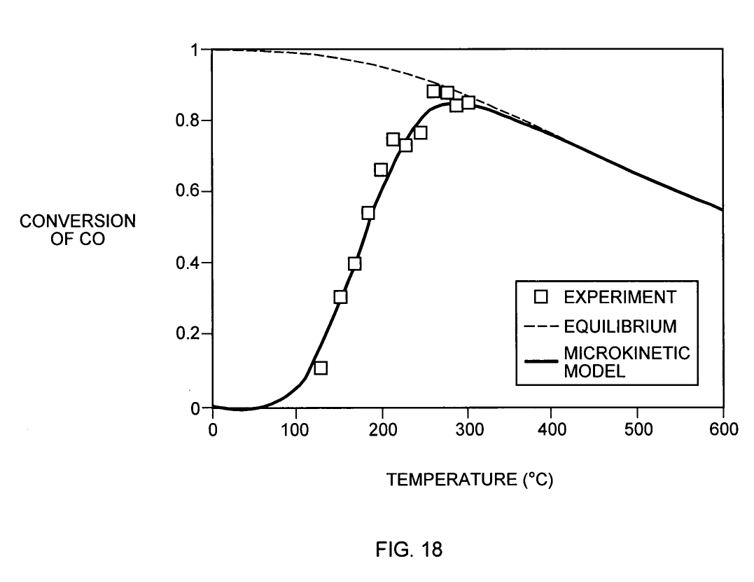

which includes the three parallel pathways and predicted rate and equilibrium constants (FIG. 12), and predicts very well the experimental kinetics (FIG. 18).

In summary, it is seen that the WGS RR graph (FIG. 14) not only affords an unmatched pictorial representation of the mechanism and the numerous possible reaction pathways, it also allows a quantitative analysis and comparison of the resistances of the various pathways (FIG. 16) based on the rates of the microkinetic model in FIG. 12, which, in turn, allows identification of the bottleneck steps and an explicit predictive rate expression.

While the above description contains much specificity, these should not be construed as limitations on the scope of the invention, but as exemplifications of the presently preferred embodiments thereof. Thus, the scope of the invention should be determined by the appended claims and their legal equivalents, and not by the examples given.

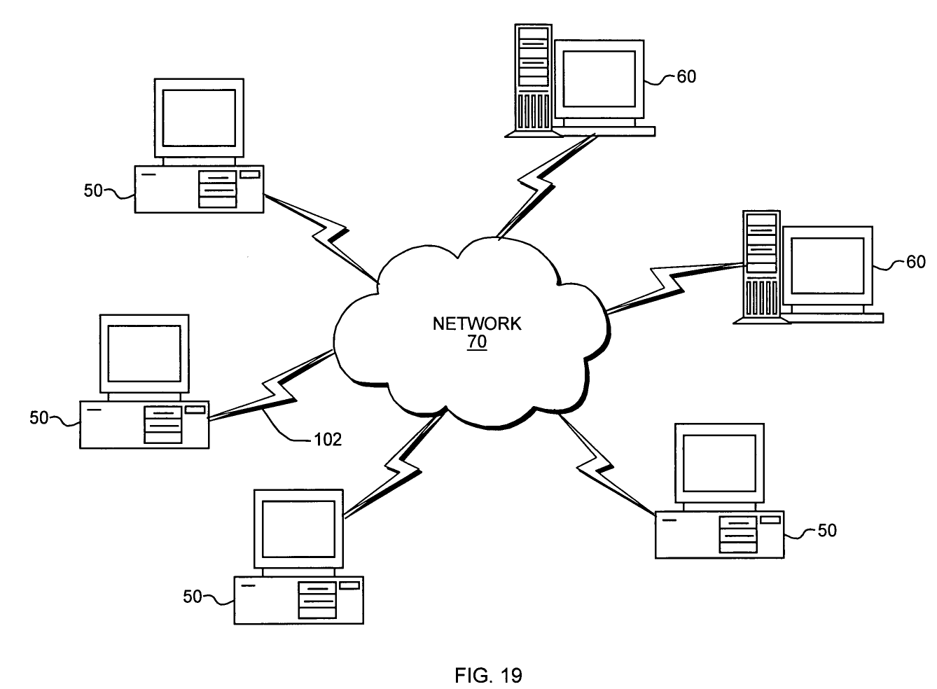

At least some embodiments of the present invention include computer implementation of methods to produce and/or analyze one or more reaction route graphs. FIG. 19 illustrates one embodiment of such a computer implementation. Client computer(s) 50 and server computer(s) 60 provide processing, storage, and input/output devices executing application programs and the like. Client computer(s) 50 can also be linked through communications network 70 to other computing devices, including other client computer(s) 50 and server computer(s) 60. Communications network 70 may be part of a local area network, a wide area network or global network (e.g., the Internet, or a worldwide collection of computers, networks, and gateways that currently use the TCP/IP suite of protocols to communicate with one another). In another embodiment of the present invention, the methods are implemented on a stand-alone computer.

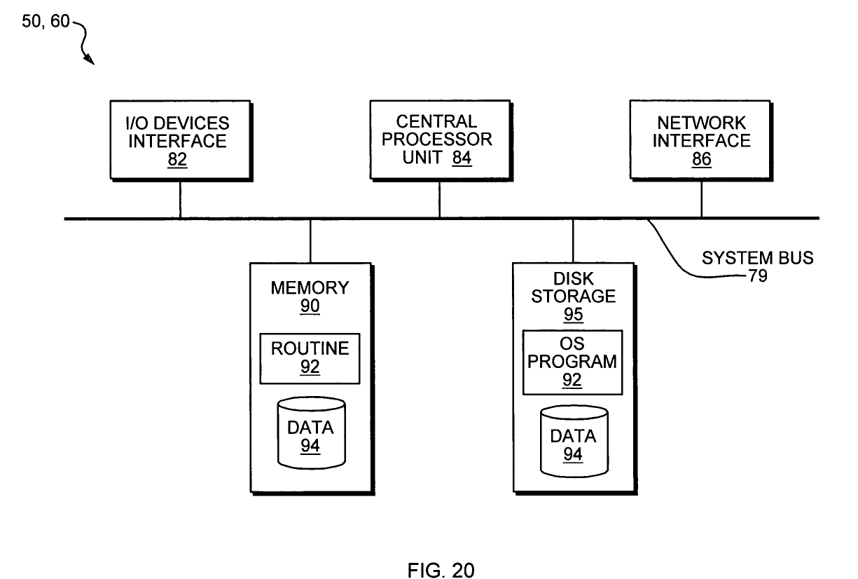

FIG. 20 is a diagram of the internal structure of a computer (e.g., client computer(s) 50 or server computers 60) in the computer system/network of FIG. 19. Each computer contains system bus 79, where a bus is a set of hardware lines used for data transfer among the components of a computer. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., displays, printers, speakers, etc.) to the computer. Network interface 86 allows the computer to connect to various other devices attached to a network (e.g., network 70 of FIG. 19). Memory 90 provides volatile storage for computer software instructions used to implement an embodiment of the present invention (e.g., Program Routines 92 and Data 94, such as Process 10). Disk storage 95 provides non-volatile storage for computer software instructions and data used to implement an embodiment of the present invention. Central processor unit 84 is also attached to system bus 79 and provides for the execution of computer instructions.

In one embodiment, computer program product 92, 94, including a computer readable medium (e.g., a removable storage medium such as one or more DVD-ROM's, CD-ROM's, diskettes, tapes, etc.) provides at least a portion of the software instructions for process 10, user interface 82, and/or any of component of computer system 50, 60. Computer program product 92, 94 can be installed by any suitable software installation procedure, as is well known in the art. In another embodiment, at least a portion of the software instructions may also be downloaded over a wireless connection. Computer program propagated signal product 102 embodied on a propagated signal on a propagation medium (e.g., a radio wave, an infrared wave, a laser wave, a sound wave, or an electrical wave propagated over a computer network such as the Internet, or other network(s)) provides at least a portion of the software instructions for invention process 10, user interface 82, and/or any component of invention computer program 92, 94.

In alternate embodiments, the propagated signal 102 is an analog carrier wave or digital signal carried on the propagated medium. For example, the propagated signal may be a digitized signal propagated over a global network (e.g., the Internet), a telecommunications network, or other network. In one embodiment, the propagated signal 102 is a signal that is transmitted over the propagation medium over a period of time, such as the instructions for a software application sent in packets over a network over a period of milliseconds, seconds, minutes, or longer. In another embodiment, the computer readable medium of computer program product 92, 94 is a propagation medium that the computer system 50, 60 may receive and read, such as by receiving the propagation medium and identifying a propagated signal embodied in the propagation medium.

While this invention has been particularly shown and described with references to preferred embodiments thereof, it will be understood by those skilled in the art that various changes in form and details may be made therein without departing from the scope of the invention encompassed by the appended claims.

1. A method for graphing chemical reaction mechanisms, comprising the steps of:
a. using a branch to represent a reaction step, either elementary or overall;
b. using nodes to represent reaction interconnectivity as well as a given group of species, wherein
i. if only intermediate species are represented, then the node is an intermediate node, and
ii. if the overall reaction is present, then the node is a terminal node;
c. enabling overall reaction routes to be traced as trails between two corresponding terminal nodes; and
d. allowing empty reaction routes to be traced as walks starting at a node, and returning to that node,
wherein at least steps c and d are computer implemented.

2. A method as claimed in claim 1 further comprising the step of constructing an energy diagram of a subject chemical reaction mechanisms by:
a. using elementary reaction energetics of the reaction mechanism, and
b. based on connectivity indicated by nodes.

3. An energy diagram constructed by the process of claim 2.

4. A reaction route graph constructed by the process of claim 1.

5. A method for analyzing the kinetics of a chemical reaction mechanism, comprising the steps of:
a. providing a subject reaction mechanism and its energetic parameters;
b. compiling a stoichiometric matrix of said subjected reaction mechanism, in a manner that dictates connectivity of elements of a corresponding RR graph;
c. from the stoichiometric matrix, enumerating overall reaction routes, empty reaction routes, intermediate nodes and terminal nodes of said subject reaction mechanism;
d. constructing an RR graph based on the enumerated overall reaction routes, empty reaction routes, intermediate nodes and terminal nodes;
e. translating the RR graph into a RR network by representing the elements of the RR graph by their respective electrical counterparts; and
f. simulating the subject reaction mechanism, equating elementary step resistances in the RR network for comparison of trails between the respective elements in the RR graph.

6. A method as in claim 5 wherein the step providing includes providing pre-exponential factors and activation energies as parameters of the subject reaction mechanism.

7. A method of claim 5 wherein the elements of the RR graph include nodes corresponding to groups of species and branches corresponding to elementary reactions of said subject reaction mechanism.

8. A method of claim 5 wherein the step of translating includes:
i. for a steady-state case, replacing a branch by a resistor, with resistance defined by Eq. [16], representing the elementary reaction step;
ii. for an unsteady-state case, replacing a branch by a resistor and capacitor, in parallel, representing the elementary step;
iii. relating branch voltage to reaction affinity, as defined by Eq. [3], either elementary or overall; and
iv. relating branch current to reaction rate, either elementary or overall.

9. A method as claimed in claim 8 wherein the steps (i) and (ii) replacing a branch include, where the branch is associated with overall reaction, replacing the branch by a power source.

10. A method as claimed in claim 8 when any combination of the steps a-e and i-iv are computer implemented.

11. A method claim 5 further comprising the steps for simplifying the reaction mechanism by:
a. reducing the RR network based on the results of the step of simulating;
b. applying Kirchhoff's Voltage Law and Kirchhoff s Current Law, in a manner where
i. Kirchhoff's Voltage Law is analogous to thermodynamic consistency and
ii. Kirchhoff's Current Law is analogous to conservation of mass; and
c. optionally deriving a simplified numerical rate expression for the overall reaction.

12. A method as claimed in claim 11 further comprising the step of constructing an energy diagram of the subject reaction mechanism by (a) using reaction energetics and (b) based on connectivity indicated by the RR graph.
